(19) United States
(12) Patent Application Publication
(10) Pub. No.: US 2025/0316345 A1
(43) Pub. Date: Oct. 9, 2025

(54) BIOMARKERS FOR ESTIMATING PHYSIOLOGICAL AGE AND RELATED METHODS, SYSTEMS, AND APPLICATIONS

(71) Applicant: PRECOGIFY PHARMACEUTICAL CHINA CO., LTD., Beijing (CN)

(72) Inventor: Kai LIN, Beijing (CN)

(73) Assignee: PRECOGIFY PHARMACEUTICAL CHINA CO., LTD., Beijing (CN)

(21) Appl. No.: 19/098,971

(22) Filed: Apr. 2, 2025

(30) Foreign Application Priority Data
Apr. 7, 2024 (CN) 202410412978.2

(51) Int. Cl. G16C 20/70; G01N 30/72; G01N 30/86

(52) U.S. Cl. CPC G16C 20/70; G01N 30/8693; G01N 30/7233

(57) ABSTRACT

Embodiments of the present disclosure provide a system, method, and application for estimating a physiological age of a subject. The system comprises: at least one storage device configured to store a set of instructions; at least one processor in communication with the at least one storage device, wherein the at least one processor is configured to perform operations when executing the set of instructions, the operations include: obtaining quantified abundance levels of one or more target metabolites from a plurality of metabolites in a sample of the subject via a quantitative measurement device; the plurality of target metabolites comprises metabolites listed in Table A; estimating the physiological age of the subject using a predictive model based on the quantified abundance levels of each of the one or more target metabolites.

### CROSS REFERENCE TO THE RELATED APPLICATIONS

This application claims priority to Chinese Patent Application No. 202410412978.2, filed on Apr. 7, 2024, the entire contents of which are incorporated herein by reference.

### TECHNICAL FIELD

The present disclosure primarily relates to the field of physiological age estimation, specifically addressing biomarkers for estimating physiological age and associated methods, systems, and applications.

### BACKGROUND

Aging involves physiological and molecular alterations, with significant variability in aging rates among individuals. Chronological age fails to comprehensively reflect aging, necessitating the concept of biological age. Reliable biomarkers of biological age are critical for individual risk stratification and anti-aging interventions.

Driven by omics technologies, next-generation tools for measuring biological aging enable quantitative characterization at molecular resolution. Epigenomic, transcriptomic, and proteomic data can be integrated with machine learning to construct “aging clocks”. However, epigenetic biomarker-based aging clocks exhibit weak associations with mortality risk, transcriptomic clocks show limited accuracy in age prediction, and proteomic clocks include proteins linked to health and longevity. These omics-derived biomarkers establish biological aging clocks in healthy populations, where deviations may indicate aging rates-positive age gaps correlate with higher mortality risks and cancer development. Concurrently, cancer tissues display age acceleration in Horvath clocks (a method assessing biological age via genomic methylation patterns).

Metabolic dysregulation is a hallmark of aging, with metabolomic biomarkers gaining attention for predicting biological aging. Plasma metabolites—such as albumin, very-low-density lipoprotein particles, and amino acids—can build metabolomic age clocks, where acceleration correlates with cardiovascular risk factors, disease susceptibility, and mortality. Additionally, replenishing age-depleted metabolites may mitigate cardiovascular pathologies. These findings suggest that metabolomic aging biomarkers may inform strategies for aging regeneration.

### SUMMARY

One or more embodiments of the present disclosure provide a system for estimating the physiological age of a subject, the system comprises: at least one storage device storing a set of instructions; at least one processor in communication with the storage device(s), wherein execution of the instructions directs the processor to perform operations including: acquiring quantified abundances of one or more target metabolites from a biological sample of the subject via a quantitative measurement device; the target metabolites comprising those listed in Table A:

TABLE A

| Number | Ion Mode | m/z | Metabolite | Exact Mass | Adduct Ion | Δ(ppm) |
|---|---|---|---|---|---|---|
| MET001 | Negative | 369.1743 | C20H22O3 | 310.15689 | M + Hac − H | 10 |
| MET002 | Negative | 369.1743 | C18H28O3S | 324.17592 | M + FA − H | 0 |
| MET003 | Negative | 369.1712 | C21H24O3 | 324.17254 | M + FA − H | 1 |
| MET004 | Negative | 371.1867 | C22H28O5 | 372.19367 | M − H | 1 |
| MET005 | Negative | 465.2453 | C25H38O8 | 466.25667 | M − H | 9 |
| MET006 | Positive | 431.3104 | C27H42O4 | 430.30831 | M + H | 12 |
| MET007 | Negative | 502.2896 | C25H46NO7P | 503.30119 | M − H | 9 |
| MET008 | Negative | 369.1743 | C22H26O5 | 370.17802 | M − H | 10 |
| MET009 | Positive | 247.1065 | C13H14N2O3 | 246.10044 | M + H | 5 |
| MET010 | Positive | 280.1540 | C15H21NO4 | 279.14706 | M + H | 1 |
| MET011 | Positive | 265.1178 | C13H16N2O4 | 264.11101 | M + H | 2 |
| MET012 | Negative | 151.0251 | C5H4N4O2 | 152.03343 | M − H | 7 |
| MET013 | Positive | 357.2781 | C24H36O2 | 356.27153 | M + H | 2 |
| MET014 | Negative | 369.1743 | C19H30O5S | 370.18140 | M − H | 1 |
| MET015 | Positive | 504.3076 | C23H43O7P | 462.27464 | M + ACN + H | 2 |

The physiological age of the subject is estimated by applying a predictive model based on the quantified abundance of each of the one or more target metabolites. In some embodiments, the one or more target metabolites include at least two, three, or ten metabolites from Table A. In some embodiments, the one or more target metabolites comprise all metabolites listed in Table A.

In certain embodiments of the present disclosure, the plurality of target metabolites further includes metabolites from Table B:

TABLE B

| Number | Ion Mode | m/z | Metabolite | Exact Mass | Adduct Ion | Δ(ppm) |
|---|---|---|---|---|---|---|
| MET016 | Positive | 287.0989 | C16H14O5 | 286.08412 | M + H | 26 |
| MET017 | Positive | 286.1432 | C17H19NO3 | 285.13649 | M + H | 2 |
| MET018 | Negative | 526.3474 | C24H52NO6P | 481.35323 | M + FA − H | 8 |
| MET019 | Negative | 367.1555 | C21H24N2O2S | 368.15585 | M − H | 19 |
| MET020 | Negative | 371.1901 | C19H32O5S | 372.19705 | M − H | 1 |
| MET021 | Negative | 365.1356 | C17H22N2O7 | 366.14270 | M − H | 1 |
| MET022 | Positive | 347.1219 | C13H25O7P | 324.13379 | M + Na | 3 |
| MET023 | Negative | 415.2165 | C24H32O6 | 416.21989 | M − H | 9 |
| MET024 | Negative | 567.3128 | C30H49O8P | 568.31651 | M − H | 6 |
| MET025 | Negative | 111.0074 | C6H5Cl | 112.00798 | M − H | 60 |
| MET026 | Negative | 371.1901 | C18H30O3S | 326.19157 | M + FA − H | 1 |
| MET027 | Positive | 288.2892 | C17H37NO2 | 287.28243 | M + H | 2 |
| MET028 | Positive | 263.1386 | C14H18N2O3 | 262.13174 | M + H | 2 |
| MET029 | Positive | 286.1433 | C17H18O4 | 286.12051 | M + NH4 − H2O | 0 |
| MET030 | Positive | 248.0696 | C8H13N3O4S | 247.06268 | M + H | 2 |
| MET031 | Negative | 203.0820 | C11H12N2O2 | 204.08988 | M − H | 3 |
| MET032 | Positive | 312.1577 | C19H21NO3 | 311.15214 | M + H | 6 |
| MET033 | Positive | 130.0498 | C5H7NO3 | 129.04259 | M + H | 1 |
| MET034 | Negative | 437.0513 | C19H19ClN2O6S | 438.06524 | M − H | 15 |
| MET035 | Negative | 528.2592 | C26H43NO8S | 529.27094 | M − H | 8 |
| MET036 | Positive | 146.0598 | C9H7NO | 145.05276 | M + H | 2 |
| MET037 | Negative | 191.0190 | C6H8O7 | 192.02700 | M − H | 4 |
| MET038 | Positive | 309.0211 | C6H14O10P2 | 308.00622 | M + H | 25 |
| MET039 | Negative | 194.0452 | C9H9NO4 | 195.05316 | M − H | 3 |
| MET040 | Negative | 399.2214 | C24H32O5 | 400.22497 | M − H | 9 |
| MET041 | Positive | 114.0915 | C6H11NO | 113.08406 | M + H | 1 |
| MET042 | Positive | 450.3207 | C21H45NO3S | 391.31202 | M + Hac − H | 11 |
| MET043 | Negative | 173.0923 | C8H14O4 | 174.08921 | M − H | 60 |
| MET044 | Positive | 205.0969 | C9H9NO2 | 163.06333 | M + ACN + H | 1 |
| MET045 | Positive | 363.2158 | C20H26O4 | 330.18311 | M + CH3OH + H | 2 |

In some embodiments, the one or more target metabolites include one metabolite from Table A and one metabolite from Table B. In some embodiments, the one or more target metabolites include one metabolite from Table A and two metabolites from Table B. In some embodiments, the one or more target metabolites include two metabolites from Table A and one metabolite from Table B.

In certain embodiments of the present disclosure, the plurality of target metabolites further comprises metabolites from Table C:

TABLE C

| Number | Ion Mode | m/z | Metabolite | Exact Mass | Adduct Ion | Δ(ppm) |
|---|---|---|---|---|---|---|
| MET046 | Negative | 367.1580 | C14H26O8 | 322.16277 | M + FA − H | 8 |
| MET047 | Negative | 399.2214 | C21H36O5S | 400.22835 | M − H | 1 |
| MET048 | Negative | 447.3120 | C21H40O6 | 388.28249 | M + Hac − H | 35 |
| MET049 | Positive | 398.3250 | C21H40O4 | 356.29266 | M + ACN + H | 4 |
| MET050 | Negative | 447.3120 | C27H44O5 | 448.31887 | M − H | 1 |
| MET051 | Negative | 447.3120 | C26H42O3 | 402.31340 | M + FA − H | 1 |
| MET052 | Positive | 442.3520 | C25H47NO5 | 441.34542 | M + H | 2 |
| MET053 | Negative | 369.1740 | C15H24N4O4 | 324.17976 | M + FA − H | 11 |
| MET054 | Negative | 367.1587 | C19H28O5S | 368.16575 | M − H | 1 |
| MET055 | Positive | 500.1700 | C26H29NO9 | 499.18423 | M + H | 43 |
| MET056 | Negative | 361.2020 | C21H30O5 | 362.20932 | M − H | 0 |
| MET057 | Negative | 319.2280 | C20H32O3 | 320.23515 | M − H | 0 |
| MET058 | Positive | 398.3260 | C23H43NO4 | 397.31921 | M + H | 1 |
| MET059 | Negative | 511.3020 | C31H44O6 | 512.31379 | M − H | 9 |
| MET060 | Negative | 448.3070 | C26H43NO5 | 449.31412 | M − H | 0 |
| MET061 | Negative | 297.9830 | C5H7NO6P2 | 238.97486 | M + Hac − H | 19 |
| MET062 | Positive | 372.3000 | C24H37NO2 | 371.28243 | M + H | 28 |
| MET063 | Negative | 173.1190 | C9H18O3 | 174.12559 | M − H | 4 |
| MET064 | Positive | 341.2320 | C19H32O5 | 340.22497 | M + H | 1 |
| MET065 | Negative | 590.3460 | C33H45N5O5 | 591.34207 | M − H | 19 |
| MET066 | Positive | 302.1960 | C16H23N5O | 301.19026 | M + H | 5 |
| MET067 | Negative | 314.1030 | C17H17NO5 | 315.11067 | M − H | 1 |
| MET068 | Positive | 355.2270 | C23H30O3 | 354.21950 | M + H | 1 |
| MET069 | Positive | 181.0720 | C7H10N4O3 | 198.07529 | M + H − H2O | 3 |
| MET070 | Positive | 180.0647 | C7H8N4O2 | 180.06473 | M+ | 0 |
| MET071 | Negative | 504.3100 | C32H43NO4 | 505.31921 | M − H | 4 |
| MET072 | Negative | 397.2057 | C23H30N2O4 | 398.22056 | M − H | 19 |
| MET073 | Positive | 355.2830 | C21H38O4 | 354.27701 | M + H | 4 |
| MET074 | Positive | 195.0870 | C8H10N4O2 | 194.08038 | M + H | 3 |
| MET075 | Negative | 427.1630 | C24H23F3N2O2 | 428.17116 | M − H | 2 |
| MET076 | Positive | 432.3110 | C25H41NO2 | 387.31373 | M + FA − H | 2 |
| MET077 | Negative | 415.2165 | C16H34O9 | 370.22028 | M + FA − H | 5 |

In some embodiments, the one or more target metabolites include at least one metabolite from Table A and at least one metabolite from Table C.

In certain embodiments of the present disclosure, the quantified abundance of each of the one or more target metabolites is determined by the quantitative measurement device using a relative quantification method or an absolute quantification method.

In some embodiments, the predictive model processes the quantified abundance of each of the one or more target metabolites to generate a sample score. In some embodiments, the sample score indicates the physiological age of the subject.

In some embodiments, the predictive model is a trained machine learning model. In some embodiments, the trained machine learning model is obtained by training an initial model using a plurality of training datasets, wherein each training dataset comprises: quantified abundances of the one or more target metabolites from a reference sample of a reference subject; a label indicating the physiological age of the reference subject.

In some embodiments, the quantitative measurement device is a liquid chromatography-mass spectrometry (LC-MS) system.

One or more embodiments of the present disclosure provide a method for estimating the physiological age of a subject, comprising:

(a) obtaining quantified abundances of one or more target metabolites from a biological sample of the subject via a quantitative measurement device, wherein the target metabolites include those listed in Table A; (b) estimating the physiological age of the subject by applying a predictive model based on the quantified abundances of the one or more target metabolites.

In some embodiments, the plurality of target metabolites further comprises metabolites from Table B, wherein the one or more target metabolites include at least one metabolite from Table A and at least one metabolite from Table B.

In some embodiments, the plurality of target metabolites further comprises metabolites from Table C, wherein the one or more target metabolites include at least one metabolite from Table A and at least one metabolite from Table C.

One or more embodiments of the present disclosure provide the use of the one or more target metabolites in the preparation of a kit for estimating the physiological age of a subject, wherein the target metabolites comprise at least one, two, three, or all metabolites from Table A.

In some embodiments, the one or more target metabolites include at least one metabolite from Table A and at least one metabolite from Table B.

In some embodiments, the one or more target metabolites include at least one metabolite from Table A and at least one metabolite from Table C.

One or more embodiments of the present disclosure provide the use of the one or more target metabolites in generating a trained machine learning model for estimating the physiological age of a subject, wherein the target metabolites comprise at least one, two, three, or all metabolites from Table A.

One or more embodiments of the present disclosure provide a kit for estimating the physiological age of a subject, the kit comprising one or more target metabolites selected from a plurality of metabolites, wherein the plurality of metabolites includes those listed in Table A.

In some embodiments, the plurality of target metabolites further comprises metabolites from Table B, wherein the one or more target metabolites include at least one metabolite from Table A and at least one metabolite from Table B.

In some embodiments, the plurality of target metabolites further comprises metabolites from Table C, wherein the one or more target metabolites include at least one metabolite from Table A and at least one metabolite from Table C.

### BRIEF DESCRIPTION OF THE DRAWINGS

The present disclosure will be further illustrated by way of exemplary embodiments, which will be described in detail with reference to the accompanying drawings. These embodiments are not limiting, and in these embodiments, the same reference numerals denote the same structures, wherein:

FIG. 1 is a schematic diagram of an application scenario of a system for estimating a subject's physiological age according to some embodiments of the present disclosure;

FIG. 2 is a block diagram of an exemplary processing device according to some embodiments of the present disclosure;

FIG. 3 is an exemplary flowchart of a method for estimating a subject's physiological age according to some embodiments of the present disclosure;

FIG. 4 is a scatter plot illustrating the prediction results of a modeling cohort in physiological age prediction performance based on 15 core metabolites detected via high-resolution metabolomics according to some embodiments of the present disclosure;

FIG. 5 is a scatter plot illustrating the prediction results of a validation cohort in physiological age prediction performance based on 15 core metabolites detected via high-resolution metabolomics according to some embodiments of the present disclosure;

FIG. 6 is a scatter plot illustrating the prediction results of a modeling cohort in physiological age prediction performance based on 45 blood metabolites strongly correlated with chronological age detected via high-resolution metabolomics according to some embodiments of the present disclosure;

FIG. 7 is a scatter plot illustrating the prediction results of a validation cohort in physiological age prediction performance based on 45 blood metabolites strongly correlated with chronological age detected via high-resolution metabolomics according to some embodiments of the present disclosure;

FIG. 8 is a scatter plot illustrating the prediction results of a modeling cohort in physiological age prediction performance based on 15 core metabolites detected via targeted metabolomics according to some embodiments of the present disclosure;

FIG. 9 is a scatter plot illustrating the prediction results of a validation cohort in physiological age prediction performance based on 15 core metabolites detected via targeted metabolomics according to some embodiments of the present disclosure;

FIG. 10 is a scatter plot illustrating the prediction results of a modeling cohort in physiological age prediction performance based on 59 blood metabolites strongly correlated with chronological age detected via targeted metabolomics according to some embodiments of the present disclosure;

FIG. 11 is a scatter plot illustrating the prediction results of a validation cohort in physiological age prediction performance based on 59 blood metabolites strongly correlated with chronological age detected via targeted metabolomics according to some embodiments of the present disclosure;

FIG. 12 is a box plot illustrating the performance of a metabolite model detected via high-resolution mass spectrometry in colorectal tumor patients according to some embodiments of the present disclosure; and

FIG. 13 is a box plot illustrating the performance of a metabolite model detected via targeted mass spectrometry in colorectal tumor patients according to some embodiments of the present disclosure.

### DETAILED DESCRIPTION

To more clearly illustrate the technical solutions of the embodiments in the present disclosure, the following briefly introduces the drawings required for describing the embodiments. Evidently, the drawings described below are merely some examples or embodiments of the present disclosure. For ordinary artisans in the field, without inventive effort, the present disclosure may be applied to other analogous scenarios based on these drawings. Unless explicitly stated or contextually evident, identical reference numerals in the drawings denote identical structures or operations.

As used in the present disclosure and the claims, unless the context clearly dictates otherwise, terms such as “a,” “an,” “one,” and/or “the” are not limited to singular forms but may encompass plural meanings. Generally, terms like “comprising” and “including” indicate the presence of explicitly identified steps and elements, which do not constitute an exhaustive enumeration. Methods or devices may also contain additional steps or elements.

After reviewing the following description and drawings, various features, characteristics, operational methods of structural components, functional relationships, part combinations, and manufacturing economics within the present disclosure may become more apparent. All such aspects form part of the present disclosure. However, it is explicitly understood that the drawings are provided solely for illustrative and descriptive purposes and are not intended to limit the scope of the disclosure. The drawings are not drawn to scale.

One or more embodiments of the present disclosure provide a set of blood metabolic biomarkers for estimating a subject's biological age, along with methods, systems, and uses for estimating biological age based on these biomarkers. The method provided in the embodiments is a non-invasive approach that utilizes blood samples (e.g., serum samples) to estimate physiological age. The method obtains quantified abundance levels of one or more target metabolites from a subject's sample via a quantitative measurement device, wherein the target metabolites include those listed in Table A. Using a predictive model, the biological age of the subject is estimated based on the quantified abundance of each target metabolite. The method requires only a minimal blood volume (≤100 μL), significantly less than tests relying on other omics-based biomarkers, enabling at-home blood sampling. This approach accurately correlates with chronological age, estimates discrepancies between biological and chronological ages in diseases (e.g., colorectal cancer), and evaluates clinical efficacy of anti-aging interventions. Additionally, its efficiency and ease of use make it suitable for monitoring individual aging states, such as accelerated or delayed aging processes.

As used herein, the term “subject” refers to any human or non-human animal. Non-human animals may include mammals (e.g., chimpanzees, apes, monkeys), livestock (e.g., cattle, sheep, pigs, goats, horses), domestic mammals (e.g., dogs, cats), and laboratory animals (e.g., mice, rats, guinea pigs). In some embodiments, the subject is a human.

The term “chronological age” (also referred to as “natural age”) denotes the age calculated based on the subject's date of birth.

The term “biological age” (or “physiological age”) refers to the developmental and functional status of a subject's organs and systems, health condition, and degree of aging. While biological age generally correlates with chronological age, discrepancies may arise. Biological age serves as an indicator of overall health, aging progression, and disease risk.

As used herein, the term “modeling cohort” includes high-resolution detection modeling cohorts and targeted modeling cohorts composed of subjects, which are used to construct biological age prediction models. The term “validation cohort” includes high-resolution detection validation cohorts and targeted validation cohorts composed of subjects, designed to evaluate the universality of developed blood metabolite panels and validate the performance of biological age prediction models.

Individuals with identical chronological ages may exhibit varying aging rates due to differences in genetic backgrounds, dietary habits, or other factors. In some embodiments, metabolites in samples from subjects of different chronological ages display distinct abundance levels. Blood samples may include serum, plasma, fingertip blood, or any combination thereof. For example, one or more target metabolites may be present in serum and referred to as “serum metabolites”.

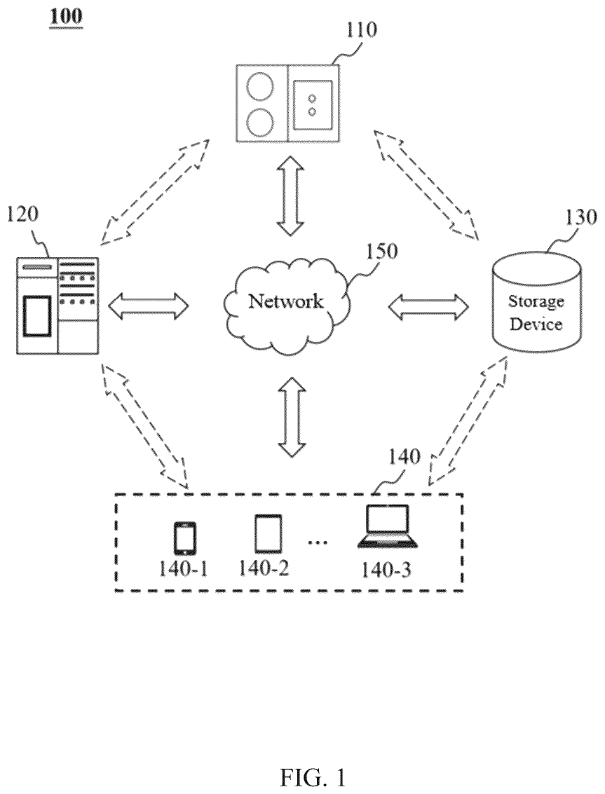

FIG. 1 illustrates an application scenario of a system for estimating a subject's biological age according to some embodiments of the present disclosure. In some embodiments, the method for estimating biological age may be implemented on System 100, which includes: Quantitative measurement device 110, Processing device 120, Storage device 130, Terminal device 140, Network 150. Connections: the quantitative measurement device 110 may connect directly to the processing device 120 (indicated by a bidirectional dashed arrow) or via network 150. The storage device 130 may connect directly to the quantitative measurement device 110 or through network 150. The terminal device 140 may link directly to the processing device 120 or via network 150.

The quantitative measurement device 110 may be configured to measure the abundance of one or more target metabolites for estimating a subject's biological age. In some embodiments, the device 110 may employ relative quantification methods or absolute quantification methods to measure metabolite abundance. For example, the device 110 may include: Mass spectrometers (MS; e.g., liquid chromatography-mass spectrometers, gas chromatography-mass spectrometers, matrix-assisted laser desorption/ionization time-of-flight mass spectrometers), Ultraviolet spectrometers, High-performance liquid chromatography (HPLC) devices.

The processing device 120 may process data and/or information obtained from the quantitative measurement device 110, storage device 130, and/or terminal device 140. In some embodiments, the processing device 120 may analyze quantified abundance levels of one or more target metabolites to estimate biological age. For example, the processing device 120 may incorporate a predictive model, where quantified metabolite abundance is input to generate a sample score indicative of the subject's biological age. The processing device 120 may determine metabolite abundance based on data acquired by the quantitative measurement device 110.

In some embodiments, the processing device 120 may be a single server or a server group. The server group may be centralized or distributed. In some embodiments, the processing device 120 may be local or remote. For example, the processing device 120 may acquire information and/or data from the quantitative measurement device 110, storage device 130, and/or terminal device 140 via the network 150. As another example, the processing device 120 may be directly connected to the quantitative measurement device 110, terminal device 140, and/or storage device 130 to access information and/or data. In some embodiments, the processing device 120 may be implemented on a cloud platform. For example, the cloud platform may include a private cloud, public cloud, hybrid cloud, community cloud, distributed cloud, cross-cloud, multi-cloud, or any combination thereof. In some embodiments, the processing device 120 may be part of the terminal device 140. In some embodiments, the processing device 120 may be part of the quantitative measurement device 110.

The storage device 130 may store data, instructions, and/or any other information. In some embodiments, it may store data acquired from the quantitative measurement device 110, processing device 120, and/or terminal device 140. This data may include quantified abundance levels of one or more target metabolites from a subject. The storage device 130 may also store data and/or instructions executable by the processing device 120 to perform methods described in the embodiments of the present disclosure. Storage Device Types: Mass storage: Disks, optical discs, solid-state drives (SSDs). Removable storage: Flash drives, floppy disks, memory cards, compact discs, magnetic tapes. Volatile read-write memory: Random access memory (RAM): Dynamic RAM (DRAM), DDR SDRAM, static RAM (SRAM), thyristor RAM (T-RAM), zero-capacitor RAM (Z-RAM). Read-only memory (ROM): Mask ROM (MROM), programmable ROM (PROM), erasable PROM (EPROM), electrically erasable PROM (EEPROM), CD-ROM, DVD-ROM. Cloud Platform Integration: The storage device 130 may be implemented on a cloud platform (e.g., private, public, hybrid, community, distributed, cross-cloud, or multi-cloud). It may connect to network 150 to communicate with other components of System 100 (e.g., processing device 120, terminal device 140). Components of System 100 may access data or instructions stored in the storage device 130 via the network 150. In some embodiments, the storage device 130 may be integrated into the quantitative measurement device 110 or processing device 120.

The terminal device 140 may connect and/or communicate with the quantitative measurement device 110, processing device 120, and/or storage device 130. In some embodiments, the terminal device 140 may include: a mobile device 140-1 (e.g., mobile phone, personal digital assistant (PDA)), a tablet computer 140-2, a laptop computer 140-3, or any combination thereof. The terminal device 140 may comprise: input devices: keyboard, touchscreen (e.g., with haptic or tactile feedback), voice input, eye-tracking input, brain monitoring system, cursor control devices (e.g., mouse, trackball, cursor direction keys). Output devices: display, printer, or any combination thereof. Functional Applications: the terminal device 140 may: present information to users, transmit user instructions to other components of System 100. Enable users to: Initiate metabolite quantification via the quantitative measurement device 110 (e.g., viewing abundance of target metabolites), review biological age estimation results of the subject.

The network 150 may include any suitable network configured to facilitate information and/or data exchange within System 100. In some embodiments, one or more components of System 100 (e.g., the quantitative measurement device 110, processing device 120, storage device 130, and terminal device 140) may transmit information and/or data to one or more other components of System 100 via the network 150.

It should be noted that the described application scenarios are provided solely for illustrative purposes and are not intended to limit the scope of the present disclosure. A person of ordinary skill in the art may implement various modifications or variations based on the descriptions contained herein. For example, the application scenarios may further incorporate a database. As another example, the scenarios may be implemented on alternative devices to achieve similar or distinct functionalities. Such modifications and variations shall not depart from the scope of the present disclosure.

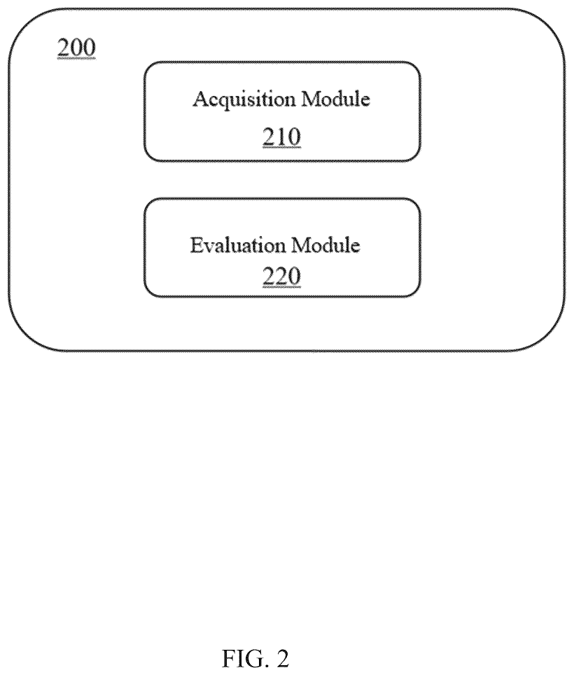

FIG. 2 illustrates an exemplary block diagram 200 of a processing device in accordance with some embodiments of the present disclosure. In some embodiments, the processing device 120 may include an acquisition module 210 and an assessment module 220. These modules may constitute entire or partial hardware circuitry of the processing device 120. Alternatively, they may be implemented as applications or instruction sets executable by the processing device 120. The modules may further represent a combination of hardware circuitry and software instructions. For example, the modules may operate as functional components of the processing device 120 while executing the instructions. In some embodiments, the processing device 120 may incorporate a processor implemented within the terminal device 140.

The acquisition module 210 may obtain quantified abundance of one or more target metabolites from a panel of multiple metabolites within a biological sample of a subject.

The assessment module 220 may: determine a sample score by processing the quantified abundance of each target metabolite through a predictive model. Estimate the physiological age of the subject based on the sample score.

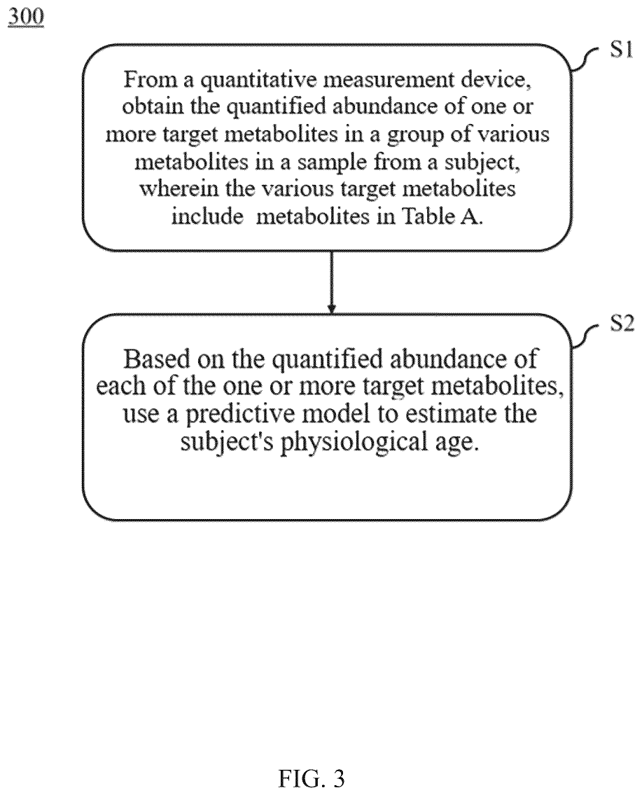

Further details regarding the acquisition module 210 and assessment module 220 are described in FIG. 3 and its associated descriptions.

It shall be understood that the system and its modules illustrated in FIG. 2 may be implemented through diverse technical configurations. It is noted that the descriptions of the processing device and its modules (e.g., acquisition module 210, assessment module 220) are provided for illustrative clarity and shall not limit the scope of the present disclosure to the disclosed embodiments. A person skilled in the art may reconfigure or functionally combine the modules (e.g., forming subsystems or redistributing interconnections) without departing from the operational principles of the system. In certain embodiments, the acquisition module 210 and assessment module 220 may operate as distinct modules within a unified system; Alternatively, a single integrated module may execute functionalities attributed to two or more modules. For example, modules may share a common storage module for data interoperability; Alternatively, each module may utilize a dedicated storage module to ensure isolated data processing. Such structural or functional adaptations fall within the protective scope of the present disclosure.

FIG. 3 illustrates an exemplary flowchart of a method for estimating the biological age of a subject according to some embodiments of the present disclosure. In some embodiments, the process 300 may be executed by a processing device 120. As shown in FIG. 3, the process 300 may include step S1 and step S2.

Step S1: Obtain quantified abundances of one or more target metabolites from a set of multiple metabolites in a sample derived from the subject via a quantitative measurement device. The multiple target metabolites include metabolites listed in Table A.

In some embodiments, the quantitative measurement device may measure the concentration or absolute quantity of metabolites in a blood sample. For example, the blood sample may include serum, plasma, or capillary blood (e.g., fingertip blood). The abundance of metabolites may be determined as a relative abundance based on normalized values or values relative to a control group. In some embodiments, the control group may comprise concentrations or quantities of the same metabolites derived from samples of subjects with known chronological ages. Alternatively, the abundance of metabolites may represent an absolute abundance directly reflecting the metabolite levels within the subject's body. In some embodiments, metabolite abundance may be obtained via mass spectrometry (MS), chromatography, or other suitable analytical techniques. Example techniques include: Mass spectrometry: Liquid chromatography-mass spectrometry (LC-MS), Gas chromatography-mass spectrometry (GC-MS), Matrix-assisted laser desorption/ionization time-of-flight mass spectrometry (MALDI-TOFMS). Ultraviolet (UV) spectroscopy, High-performance liquid chromatography (HPLC).

The quantitative measurement device refers to an apparatus for measuring metabolite abundance. In some embodiments, the device may include a mass spectrometer (MS), ultraviolet (UV) spectrophotometer, or high-performance liquid chromatography (HPLC) system.

In some embodiments, the quantitative measurement device may be configured as a liquid chromatography-mass spectrometry (LC-MS) hyphenated system.

In some embodiments, the quantitative measurement device may employ either a relative quantification method or an absolute quantification method to measure the abundance of one or more target metabolites.

The multiple target metabolites are blood metabolites utilized to estimate the biological age of the subject. In some embodiments, the one or more target metabolites include those listed in Table A.

Table A displays an exemplary metabolite panel applicable for estimating biological age. As biomarkers, each metabolite within the panel demonstrates a statistically robust correlation with an individual's chronological age. In some embodiments, the biomarkers for biological age estimation disclosed herein may comprise one or more target metabolites listed in Table A.

In some embodiments, one or more metabolites shown in Table A can be utilized to estimate the physiological age of a subject. For example, mass spectrometry (or other analytical techniques) may be employed to quantify the abundance of one or more target metabolites among a panel of multiple metabolites in a sample. The quantified abundance of each metabolite can be processed and applied to estimate the physiological age of the subject. In certain embodiments, any single metabolite from Table A may be quantified and utilized for such purposes. In further embodiments, any two, three, or ten metabolites from Table A may be quantified and employed for these objectives. In specific embodiments, all metabolites in Table A may be quantified and applied for the aforementioned purposes.

TABLE A

| | Ion | | | Exact | Adduct | |
|---|---|---|---|---|---|---|
| Number | Mode | m/z | Metabolite | Mass | Ion | Δ(ppm) |
| MET001 | Negative | 369.1743 | C20H22O3 | 310.15689 | M + Hac − H | 10 |
| MET002 | Negative | 369.1743 | C18H28O3S | 324.17592 | M + FA − H | 0 |
| MET003 | Negative | 369.1712 | C21H24O3 | 324.17254 | M + FA − H | 1 |
| MET004 | Negative | 371.1867 | C22H28O5 | 372.19367 | M − H | 1 |
| MET005 | Negative | 465.2453 | C25H38O8 | 466.25667 | M − H | 9 |
| MET006 | Positive | 431.3104 | C27H42O4 | 430.30831 | M + H | 12 |
| MET007 | Negative | 502.2896 | C25H46NO7P | 503.30119 | M − H | 9 |
| MET008 | Negative | 369.1743 | C22H26O5 | 370.17802 | M − H | 10 |
| MET009 | Positive | 247.1065 | C13H14N2O3 | 246.10044 | M + H | 5 |
| MET010 | Positive | 280.1540 | C15H21NO4 | 279.14706 | M + H | 1 |
| MET011 | Positive | 265.1178 | C13H16N2O4 | 264.11101 | M + H | 2 |
| MET012 | Negative | 151.0251 | C5H4N4O2 | 152.03343 | M − H | 7 |
| MET013 | Positive | 357.2781 | C24H36O2 | 356.27153 | M + H | 2 |
| MET014 | Negative | 369.1743 | C19H30O5S | 370.18140 | M − H | 1 |
| MET015 | Positive | 504.3076 | C23H43O7P | 462.27464 | M + ACN + H | 2 |

In some embodiments, the one or more target metabolites comprise at least two, three, or ten metabolites from Table A. In certain embodiments, the one or more target metabolites may encompass all metabolites in Table A.

In some embodiments, the one or more target metabolites utilized as biomarkers for estimating the physiological age of a subject further comprise metabolites from Table B.

TABLE B

| | Ion | | | Exact | Adduct | |
|---|---|---|---|---|---|---|
| Number | Mode | m/z | Metabolite | Mass | Ion | Δ(ppm) |
| MET016 | Positive | 287.0989 | C16H14O5 | 286.08412 | M + H | 26 |
| MET017 | Positive | 286.1432 | C17H19NO3 | 285.13649 | M + H | 2 |
| MET018 | Negative | 526.3474 | C24H52NO6P | 481.35323 | M + FA − H | 8 |
| MET019 | Negative | 367.1555 | C21H24N2O2S | 368.15585 | M − H | 19 |
| MET020 | Negative | 371.1901 | C19H32O5S | 372.19705 | M − H | 1 |
| MET021 | Negative | 365.1356 | C17H22N2O7 | 366.14270 | M − H | 1 |
| MET022 | Positive | 347.1219 | C13H25O7P | 324.13379 | M + Na | 3 |
| MET023 | Negative | 415.2165 | C24H32O6 | 416.21989 | M − H | 9 |
| MET024 | Negative | 567.3128 | C30H49O8P | 568.31651 | M − H | 6 |
| MET025 | Negative | 111.0074 | C6H5Cl | 112.00798 | M − H | 60 |
| MET026 | Negative | 371.1901 | C18H30O3S | 326.19157 | M + FA − H | 1 |
| MET027 | Positive | 288.2892 | C17H37NO2 | 287.28243 | M + H | 2 |
| MET028 | Positive | 263.1386 | C14H18N2O3 | 262.13174 | M + H | 2 |
| MET029 | Positive | 286.1433 | C17H18O4 | 286.12051 | M + NH4 − H2O | 0 |
| MET030 | Positive | 248.0696 | C8H13N3O4S | 247.06268 | M + H | 2 |
| MET031 | Negative | 203.0820 | C11H12N2O2 | 204.08988 | M − H | 3 |
| MET032 | Positive | 312.1577 | C19H21NO3 | 311.15214 | M + H | 6 |
| MET033 | Positive | 130.0498 | C5H7NO3 | 129.04259 | M + H | 1 |
| MET034 | Negative | 437.0513 | C19H19ClN2O6S | 438.06524 | M − H | 15 |
| MET035 | Negative | 528.2592 | C26H43NO8S | 529.27094 | M − H | 8 |
| MET036 | Positive | 146.0598 | C9H7NO | 145.05276 | M + H | 2 |
| MET037 | Negative | 191.0190 | C6H8O7 | 192.02700 | M − H | 4 |
| MET038 | Positive | 309.0211 | C6H14O10P2 | 308.00622 | M + H | 25 |
| MET039 | Negative | 194.0452 | C9H9NO4 | 195.05316 | M − H | 3 |
| MET040 | Negative | 399.2214 | C24H32O5 | 400.22497 | M − H | 9 |
| MET041 | Positive | 114.0915 | C6H11NO | 113.08406 | M + H | 1 |
| MET042 | Positive | 450.3207 | C21H45NO3S | 391.31202 | M + Hac − H | 11 |
| MET043 | Negative | 173.0923 | C8H14O4 | 174.08921 | M − H | 60 |
| MET044 | Positive | 205.0969 | C9H9NO2 | 163.06333 | M + ACN + H | 1 |
| MET045 | Positive | 363.2158 | C20H26O4 | 330.18311 | M + CH3OH + H | 2 |

In some embodiments, the one or more target metabolites comprise one metabolite from Table A and one metabolite from Table B. In certain embodiments, the one or more target metabolites include one metabolite from Table A and two metabolites from Table B. In further embodiments, the one or more target metabolites include two metabolites from Table A and one metabolite from Table B. Any combination of one or more metabolites from Table A and one or more metabolites from Table B may be utilized to achieve the same objective. In specific embodiments, one or more metabolites from Table B may be employed independently of the metabolites listed in Table A to estimate the physiological age of the subject.

In some embodiments, the one or more target metabolites utilized as biomarkers for estimating the physiological age of a subject further comprise metabolites from Table C.

TABLE C

| | Ion | | | Exact | Adduct | |
|---|---|---|---|---|---|---|
| Number | Mode | m/z | Metabolite | Mass | Ion | Δ(ppm) |
| MET046 | Negative | 367.1580 | C14H26O8 | 322.16277 | M + FA − H | 8 |
| MET047 | Negative | 399.2214 | C21H36O5S | 400.22835 | M − H | 1 |
| MET048 | Negative | 447.3120 | C21H40O6 | 388.28249 | M + Hac − H | 35 |
| MET049 | Positive | 398.3250 | C21H40O4 | 356.29266 | M + ACN + H | 4 |
| MET050 | Negative | 447.3120 | C27H44O5 | 448.31887 | M − H | 1 |
| MET051 | Negative | 447.3120 | C26H42O3 | 402.31340 | M + FA − H | 1 |
| MET052 | Positive | 442.3520 | C25H47NO5 | 441.34542 | M + H | 2 |
| MET053 | Negative | 369.1740 | C15H24N4O4 | 324.17976 | M + FA − H | 11 |
| MET054 | Negative | 367.1587 | C19H28O5S | 368.16575 | M − H | 1 |
| MET055 | Positive | 500.1700 | C26H29NO9 | 499.18423 | M + H | 43 |
| MET056 | Negative | 361.2020 | C21H30O5 | 362.20932 | M − H | 0 |
| MET057 | Negative | 319.2280 | C20H32O3 | 320.23515 | M − H | 0 |
| MET058 | Positive | 398.3260 | C23H43NO4 | 397.31921 | M + H | 1 |
| MET059 | Negative | 511.3020 | C31H44O6 | 512.31379 | M − H | 9 |
| MET060 | Negative | 448.3070 | C26H43NO5 | 449.31412 | M − H | 0 |
| MET061 | Negative | 297.9830 | C5H7NO6P2 | 238.97486 | M + Hac − H | 19 |
| MET062 | Positive | 372.3000 | C24H37NO2 | 371.28243 | M + H | 28 |
| MET063 | Negative | 173.1190 | C9H18O3 | 174.12559 | M − H | 4 |
| MET064 | Positive | 341.2320 | C19H32O5 | 340.22497 | M + H | 1 |
| MET065 | Negative | 590.3460 | C33H45N5O5 | 591.34207 | M − H | 19 |
| MET066 | Positive | 302.1960 | C16H23N5O | 301.19026 | M + H | 5 |
| MET067 | Negative | 314.1030 | C17H17NO5 | 315.11067 | M − H | 1 |
| MET068 | Positive | 355.2270 | C23H30O3 | 354.21950 | M + H | 1 |
| MET069 | Positive | 181.0720 | C7H10N4O3 | 198.07529 | M + H − H2O | 3 |
| MET070 | Positive | 180.0647 | C7H8N4O2 | 180.06473 | M+ | 0 |
| MET071 | Negative | 504.3100 | C32H43NO4 | 505.31921 | M − H | 4 |
| MET072 | Negative | 397.2057 | C23H30N2O4 | 398.22056 | M − H | 19 |
| MET073 | Positive | 355.2830 | C21H38O4 | 354.27701 | M + H | 4 |
| MET074 | Positive | 195.0870 | C8H10N4O2 | 194.08038 | M + H | 3 |
| MET075 | Negative | 427.1630 | C24H23F3N2O2 | 428.17116 | M − H | 2 |
| MET076 | Positive | 432.3110 | C25H41NO2 | 387.31373 | M + FA − H | 2 |
| MET077 | Negative | 415.2165 | C16H34O9 | 370.22028 | M + FA − H | 5 |

In certain embodiments, the one or more target metabolites comprise at least one metabolite from Table A and at least one metabolite from Table C.

In further embodiments, the one or more target metabolites may include one or more metabolite combinations shown in Table C, excluding any metabolites listed in Table A. Alternatively, the one or more target metabolites may include one or more metabolite combinations from Table C and at least one metabolite selected from Table A.

It should be noted that one or more metabolites listed in Tables A, B, and C may exist in one or more isomeric forms, all of which are encompassed within the scope of the diagnostic biomarker panel provided by the embodiments of the present disclosure.

Step S2: Based on the quantified abundance of each target metabolite within the one or more target metabolites, a predictive model is employed to estimate the physiological age of the subject.

The physiological age predictive model (or simply referred to as the predictive model) is a machine learning model configured to determine the physiological age of the subject.

In some embodiments, the predictive model may be a trained machine learning model. Exemplary machine learning models include, but are not limited to: Weighted regression models. Linear regression models. Elastic net regression models. Random forest models. Neural network models, or any combination thereof. For example, after calculating correlations between blood metabolites and calendar age using common coefficient methods (e.g., Pearson correlation coefficient) to identify blood metabolites with statistically significant correlations, the predictive model may be constructed based on calendar age by employing algorithms such as: Weighted regression algorithms, Linear regression algorithms, Elastic net regression algorithms, Random forest algorithms, Neural networks.

In some embodiments, the predictive model processes the quantified abundance of each of the one or more target metabolites to determine a sample score. For example, Step S2 may include normalizing the abundance of each metabolite quantified in Step S1 and determining the sample score by processing the normalized abundances with the predictive model.

In certain embodiments, the determination of the sample score may be implemented on a computing device (e.g., the processing device 120 shown in FIG. 1). The computing device may acquire the predictive model for determining the sample score. The abundance of each metabolite quantified in Step S1 may be input into the predictive model. The predictive model may process the abundance of each metabolite quantified in Step S1 (e.g., relative abundance or absolute abundance) and output the sample score. As an example, the abundance of each metabolite quantified in Step S1 may be determined by measuring the concentration of each metabolite. In some embodiments, the measured concentrations may be normalized (e.g., by dividing the measured concentrations by the total concentration of all metabolites in the sample).

In further embodiments, the sample score indicates the physiological age of the subject. For instance, a sample score of 55 corresponds to an estimated physiological age of 55 years for the subject.

To develop the predictive model, multiple training datasets may be utilized for preliminary model training.

In some embodiments, each of the multiple training datasets may comprise: quantified abundances of one or more target metabolites in reference samples from reference subjects, labels indicating the physiological age of the reference subjects. In certain embodiments, the reference subjects may include a healthy population across various age groups. The labels may correspond to the biological age of the healthy population at respective age stages. For example, multiple first training samples with first labels may be input into the predictive model. A loss function may be constructed based on the first labels and the predictive model's outputs. Parameters of the predictive model may be iteratively updated according to the loss function. Training of the predictive model is completed when: The loss function converges. The number of iterations reaches a predefined threshold. At this stage, the trained target predictive model is obtained.

In some embodiments, performance of the predictive model may be evaluated using multiple metrics. Exemplary evaluation metrics include, but are not limited to: Root Mean Square Error (RMSE), Mean Absolute Error (MAE), R-squared (R²), Correlation coefficient (R), or any combination thereof.

The Root Mean Square Error (RMSE) is a widely used metric for quantifying prediction errors. RMSE calculates the deviation between predicted values and ground-truth values, where lower RMSE values indicate closer alignment between predictions and actual values, thereby reflecting superior model performance.

The Mean Absolute Error (MAE) measures the average absolute difference between predicted and true values. Key distinctions: MAE computes the mean of absolute differences. RMSE computes the square root of the mean of squared differences. Smaller MAE values generally signify better model accuracy.

R-squared is a regression analysis metric that quantifies the proportion of variance explained by independent variables. Characteristics: Ranges between 0 and 1. Higher values denote stronger model fit. Note: R-squared alone is insufficient for comprehensive model evaluation; additional factors such as model stability and predictive capability must be considered.

Correlation analysis is a statistical method for studying relationships between variables. Common methods include:

Pearson Correlation Coefficient: Measures linear correlation between two continuous variables. Ranges from—1 (perfect negative correlation) to 1 (perfect positive correlation). 0 indicates no linear relationship.

Spearman Correlation Coefficient: Applicable to non-normally distributed or ordinal data. Evaluates monotonic relationships based on rank orders.

One or more embodiments of the present disclosure provide a method for estimating the physiological age of a subject, which may be executed by the processing device 120 shown in FIG. 1 and/or one or more modules in FIG. 2.

In some embodiments, the method may predict an individual's aging degree. Physiological age levels in populations under different disease states, based on blood metabolite detection, serve as an effective monitoring tool for aging status, revealing accelerated or delayed aging processes. Example: The method estimates the physiological age of cancer patients (e.g., colorectal cancer).

In certain embodiments, the method evaluates the efficacy of anti-aging interventions (e.g., dietary or pharmaceutical interventions) and provides a reliable, convenient clinical tool with high auxiliary value.

One or more embodiments disclose the use of one or more target metabolites in preparing a kit for estimating physiological age. The target metabolites include at least one, two, three, or all metabolites listed in Table A.

In some embodiments, the target metabolites comprise at least one metabolite from Table A and at least one from Table B.

In further embodiments, the target metabolites include at least one metabolite from Table A and at least one from Table C. For details, refer to Tables A, B, and C in FIG. 3.

One or more embodiments of the present disclosure provide one or more target metabolites for training a machine learning model to estimate the physiological age of a subject. In some embodiments, the target metabolites include at least one, two, three, or all metabolites listed in Table A. For additional details, refer to Table A in FIG. 3 and related descriptions.

In some embodiments, a kit for estimating the physiological age of a subject may comprise one or more target metabolites. The plurality of target metabolites may include metabolites listed in Table A.

In certain embodiments, the plurality of target metabolites includes metabolites from Table B. A combination of the one or more target metabolites may comprise at least one metabolite from Table A and at least one metabolite from Table B.

In some embodiments, the plurality of target metabolites comprises metabolites listed in Table C. In certain embodiments, the one or more target metabolites include at least one metabolite from Table A and at least one metabolite from Table C. For further details regarding the one or more target metabolites, refer to Tables A, B, and C in FIG. 3 and associated content.

In some embodiments, the one or more target metabolites may serve as reference materials. The reference materials may be used to determine the abundance of the one or more target metabolites in a subject. Specifically, the reference materials may be employed to generate one or more standard curves for quantifying the abundance of the one or more target metabolites in the subject. Additionally, the kit may further comprise other components not limited by the embodiments of the present disclosure, such as one or more quality control agents, one or more pre-treatment agents for processing a subject's sample (e.g., serum samples), or any combination thereof.

The method for estimating a subject's physiological age provided by embodiments of the present disclosure comprises but is not limited to the following advantages:

By employing mass spectrometry-based detection methods to analyze the relative abundance of thousands of signals in human blood, the approach achieves high signal quantity, target specificity, and simplified pre-processing of blood samples, with short analytical time, making it suitable for high-throughput analytical testing of clinical samples.

The required blood volume is exceptionally small (≤100 μL), significantly lower than tests based on other omics biomarkers. This enables at-home blood sampling, eliminating dependency on clinical facilities and facilitating direct consumer accessibility.

Human blood samples, compared to tissue samples, are more readily obtainable and psychologically acceptable to test populations versus urine or fecal samples. The method avoids radiation exposure from imaging diagnostics and demonstrates enhanced patient compliance.

The metabolite biomarkers and methods described in these embodiments are further detailed below and should not be construed as limiting the scope of the present disclosure. Performance metrics of exemplary predictive models based on the one or more target metabolites are also provided in subsequent embodiments.

### EMBODIMENTS

### Material and Method

### 1. Study Cohort

### (1) Modeling Cohort

The modeling cohort comprises: a high-resolution detection modeling cohort with 102 subjects aged 44-73 years, and a targeted detection modeling cohort with 300 subjects aged 40-74 years. Table 1 summarizes the age distribution of the modeling cohorts. Participants in the modeling cohort underwent rigorous screening to exclude confounding factors that may interfere with the identification of aging biomarkers, including: underlying health conditions, and medication usage. Informed consent was obtained from all participants.

TABLE 1
Age Distribution of Modeling Cohort

| | | Age Distribution | | | | |
|---|---|---|---|---|---|---|
| | | | Mean ± Standard | | | |
| | | Number | Deviation | Minimum | Median | Maximum |
| High-resolution Detection | Total | 102 | 58.58 ± 7.86 | 44 | 59 | 73 |
| Validation Cohort | Male | 50 | 57.16 ± 7.79 | 44 | 58.5 | 73 |
| | Female | 52 | 59.94 ± 7.77 | 45 | 59.5 | 73 |
| Targeted Detection | Total | 300 | 54.13 ± 9.2 | 40 | 53 | 74 |
| Validation Cohort | Male | 148 | 53.83 ± 8.92 | 40 | 53 | 74 |
| | Female | 152 | 54.41 ± 9.49 | 40 | 53.5 | 73 |

### (2) Validation Cohort

To evaluate the generalizability of the developed serum metabolite panel and validate model performance, validation cohorts were recruited for each metabolomics panel, comprising: a high-resolution detection validation cohort with 26 subjects aged 46-74 years, and a targeted validation cohort with 75 subjects aged 40-73 years, as detailed in Table 2.

TABLE 2
Age Distribution of Validation Cohort

| | | Age Distribution | | | | |
|---|---|---|---|---|---|---|
| | | | Mean ± Standard | | | |
| | | Number | Deviation | Minimum | Median | Maximum |
| High-resolution Detection | Total | 26 | 58.85 ± 8.18 | 46 | 59.5 | 74 |
| Validation Cohort | Male | 12 | 57.58 ± 9.11 | 46 | 57.5 | 70 |
| | Female | 14 | 59.93 ± 7.46 | 49 | 59.5 | 74 |
| Targeted Detection | Total | 75 | 53.96 ± 9.57 | 40 | 54 | 73 |
| Validation Cohort | Male | 36 | 53.33 ± 8.33 | 40 | 53.5 | 67 |
| | Female | 39 | 54.54 ± 10.67 | 40 | 54 | 73 |

### 2. Equipment, Reagents & Solutions

### (1) Equipment

Vortex Mixer (Kylin-Bell Vortex X5)
20 μL, 100 μL, 200 μL, 1000 μL pipettes with pipette tips (Gilson)
High-Speed Centrifuge (Centrifuge 5415R)
Electronic Balance (MettlerToledo AB104)
Centrifugal Vacuum Evaporator (TOMY CC-105)
Exion-20adxr Ultra-High-Performance Liquid Chromatography System (shimadzu) coupled with Triple Quad™ 4500MD LC-MS/MS System (AB Sciex)
ACQUITY UPLC BEH C18 Column (Shim-pack Velox C18 2.7 μm 2.1×100 mm)
R Statistical Scripting Language (Version 3.6.1)
AB Sciex Analyst Software System (Version 1.6.3)
Q Exactive Plus Mass Spectrometer equipped with UltiMate 3000 LC Series (ThermoFisher)
CORTECS (Waters) 1.6 μm C18+2.1*100 mm Column

### (2) Reagents and Consumables

LC-MS grade methanol (Thermo Fisher Scientific) LC-MS grade acetonitrile (Thermo Fisher Scientific)
LC-MS grade formic acid (Thermo Fisher Scientific)
LC-MS grade ammonium acetate (Thermo Fisher Scientific)
13C-labeled cholic acid (Sigma-Aldrich)
Ultra-pure water, HPLC grade (watsons)
Centrifuge tube (1.5 mL; Axygen, Model MCT-150-C)
10 μL, 200 μL, 1000 μL pipette tips (Axygen)

### (3) Solutions

13C-labeled cholic acid stock solution (internal standard): 10.8 mg of cholic acid was weighed, dissolved in 1080 μL of methanol, and vortex-mixed vigorously until complete dissolution. The final concentration of the stock solution was 10 mg/mL.

Precipitation solution: 300 mL of methanol was combined with 120 L of the 13C-labeled cholic acid stock solution and mixed thoroughly.

### 3. High-Resolution Metabolomics Detection

### (1) Metabolite Extraction

All samples were extracted via salting-out methods: 60 μL of serum was combined with 6 μL of internal standard solution (L-Tyrosine-(phenyl-3,5-d2) (100 μg/mL, Sigma-Aldrich); 13C-cholic acid (10 μg/mL, Cambridge Isotope Laboratories); Doxercalciferol (60 μg/mL, MedChem Express). 240 μL of acetonitrile (3:1, ThermoFisher) and 60 μL of ammonium formate (0.5 g/mL) were added, followed by vigorous vortex-mixing. The mixture was centrifuged at 18,000×g for 5 min. A 200 μL aliquot of the supernatant was dried using a CentriVap Cold Trap Centrifugal Evaporator (Labconco) and further centrifuged at 13,000 rpm/min for 3 min at room temperature. The residue was reconstituted in 75 μL of 55% methanol (containing 0.1% formic acid, ThermoFisher). Q Exactive Plus Mass Spectrometer (ThermoFisher) coupled with an UltiMate 3000 LC Series (ThermoFisher), operated in HESI mode (130-1200 m/z) under both positive and negative ionization. CORTECS (Waters) 1.6 μm C18+ (2.1×100 mm), maintained at 35° C. Flow Rate 0.3 mL/min; injection volume 5 μL. Mobile Phase A: Acetonitrile with 0.1% formic acid (gradient: 5%→45% from 0.5-14 min; 75% at 32 min; 80% at 42 min; 100% at 50-55 min; return to 5% within 5 min). Mobile Phase B: Ultrapure water (Merck Millipore) with 0.1% formic acid. Raw metabolomic data were preprocessed and normalized using XC-MS. Metabolites with average abundance >5,000 in UC, CD, or normal groups were included for omics analysis. Significantly differential metabolites were scanned in SIM/PRM and Full MS/dd-MS2 modes via Compound Discoverer (v3.1), referencing HMDB, mzCloud, and Chemspider databases.

### (2) Quality Control

Equal volumes (15 μL) of serum from each individual in the discovery cohort were pooled to create mixed samples serving as quality control (QC) samples. Each detection batch contained a minimum of 15 QC samples. Prior to subsequent analyses, all individual metabolite peak areas were normalized to the same QC samples.

### 4. Targeted Metabolomics Detection

### (1) Metabolite Extraction

During metabolite extraction in targeted metabolomics analysis, 10 μL of internal standard solution (5 μg/mL 13C-cholic acid) was added to 80 μL serum, followed by addition of 150 μL acetonitrile: isopropanol (4:1, v/v, Thermo Fisher) and 50 μL ammonium formate (0.5 g/mL). The mixture was vortex-mixed and centrifuged at 17,949 g for 5 minutes. Subsequently, 60 μL supernatant was collected and diluted with 150 μL HPLC-grade water prior to use.

### (2) Detection Methods

A pseudo-targeted method relying on pure standards, referencing the method from Fujian Chen et. al (Nature Protocols, 2020), determines the relative levels of all metabolites in identified panels by normalizing individual metabolite abundances using identical reference pool samples. Targeted metabolomics analysis was performed on an AB SCIEX Triple Quad™ 4500 system and operated in positive and negative modes. The mobile phases and columns for reversed-phase liquid chromatography are shown in the tables. The injection volume for each mode was 15 μL. Metabolites were eluted from the column at a flow rate of 0.3 mL/min, with the concentration of mobile phase B gradually increasing from an initial 12% to 60% after 2.5 minutes. Linear gradients of phase B from 60% to 85% and 85% to 100% were set at 6 min and 8.5 min, respectively. Declustering potentials and collision energies were optimized using quality control samples from the control group. Metabolite peaks were integrated using Sciex Analyst 1.6.3 software. Detailed parameters for chromatography and mass spectrometry refer to Feng Chen et. al (Gut Microbiota, 2021).

### (3) Quality Control

Equal volumes (15 μL) of serum from each individual in the cohort were pooled, and the mixed sample served as QC samples. Each testing batch contained at least 6 QC samples. Prior to subsequent analyses, metabolite peak areas from all individuals were normalized to the same QC samples.

### 5. Metabolomics Data Processing

Data processing was performed using the R language (version 3.6.1). To enhance data consistency and comparability, raw metabolite abundances were normalized through a locally weighted regression method. Subsequently, relative abundances were determined by calculating the ratio of each metabolite to the abundance observed in the shared QC samples. These normalized and ratio-transformed values, referred to as relative abundances, were employed in all subsequent analytical procedures, ensuring a standardized basis for exploring molecular associations and constructing predictive models within the study context.

### 6. Physiological Age Model Training and Validation

Serum metabolomics data from the modeling cohort were used to construct predictive models (e.g., linear regression models). Multiple metrics were employed to evaluate the accuracy of the age prediction models, including: Mean Absolute Error (MAE), defined as the average of absolute differences between predicted ages and chronological ages; Root Mean Square Error (RMSE), calculated as the square root of the average squared differences between predicted and chronological ages; Coefficient of Determination (R-squared), determined by the ratio of residual sum of squares to total sum of squares. Additionally, predicted phenotypic ages were compared with actual chronological ages using the cor ( ) function in R, such as by computing the Pearson correlation coefficient. Models derived from the modeling cohort were tested on the validation cohort.

### Embodiment 1 Detection of Serum Metabolite Abundance Using High-Performance Liquid Chromatography-Mass Spectrometry Methodology

High-resolution mass spectrometry-based metabolomic analysis and targeted mass spectrometry-based metabolomic analysis were performed using the high-performance liquid chromatography-mass spectrometry (HPLC-MS) detection method. Equal volumes of serum samples were mixed as quality control (QC) samples to standardize the accuracy and reproducibility of each batch. Low-abundance signals (average abundance <50,000) and inaccurate signals (coefficient of variation %>30% in QC samples) were filtered out. High-resolution mass spectrometry detected 2,410 metabolites in serum samples from 128 healthy subjects, while targeted metabolomic analysis identified 285 blood metabolites in serum samples from 375 subjects. Consequently, a system comprising 2,410 high-resolution mass spectrometry-detected blood metabolite groups and 285 targeted mass spectrometry-detected blood metabolite groups was established for detecting abundance in serum samples.

### Embodiment 2 Identification of Blood Metabolites Associated with Chronological Age

First, blood metabolites associated with chronological age in the modeling cohort were systematically identified. By analyzing blood metabolites from high-resolution mass spectrometry-based metabolomics and targeted mass spectrometry-based metabolomics, and performing Spearman correlation analysis after adjusting for the confounding factor of sex, 45 high-resolution mass spectrometry-detected blood metabolite groups and 59 targeted mass spectrometry-detected blood metabolite groups were screened, showing strong correlations with chronological age. Among these, 15 metabolite groups coexisting in both high-resolution mass spectrometry and targeted mass spectrometry datasets were identified as core metabolite groups, termed core metabolites. The random forest approach was then used to rank the importance of the 45 high-resolution-detected metabolites and 59 targeted-detected metabolites, respectively, as shown in the table below.

TABLE 3
High-Resolution Mass Spectrometry-Detected Blood Metabolites Associated
with Chronological Age in Modeling Cohort and Validation Cohort

| | | Correlation with | | | Relative Abundance | | Relative Abundance | |
|---|---|---|---|---|---|---|---|---|
| | | Chronological Age | | | in Modeling Cohort | | in Validation Cohort | |
| | Metabolite | Correlation | p- | | | Standard | | Standard |
| Importance | ID | Coefficient | Value | FDR | Mean | Deviation | Mean | Deviation |
| 1 | MET001 | −0.42 | 1.04E−06 | 2.69E−05 | 1.0300 | 0.6491 | 0.99480 | 0.78880 |
| 2 | MET002 | −0.41 | 1.79E−06 | 2.69E−05 | 1.0570 | 0.7450 | 0.97260 | 0.80110 |
| 3 | MET003 | −0.41 | 1.45E−06 | 2.69E−05 | 1.2820 | 2.4580 | 1.25100 | 1.67600 |
| 4 | MET004 | −0.38 | 1.17E−05 | 8.77E−05 | 1.1340 | 0.6458 | 1.17700 | 0.64880 |
| 5 | MET005 | −0.34 | 9.80E−05 | 3.39E−04 | 1.2030 | 0.7005 | 1.09300 | 0.53290 |
| 6 | MET006 | 0.28 | 1.37E−03 | 1.86E−03 | 1.4290 | 0.7080 | 1.22700 | 0.52260 |
| 7 | MET007 | 0.36 | 2.69E−05 | 1.73E−04 | 0.9735 | 0.4163 | 0.95840 | 0.51310 |
| 8 | MET008 | −0.38 | 8.65E−06 | 7.79E−05 | 1.0360 | 0.7347 | 1.04700 | 0.86550 |
| 9 | MET009 | 0.32 | 2.39E−04 | 5.12E−04 | 1.2990 | 0.9580 | 1.25800 | 0.93560 |
| 10 | MET010 | 0.35 | 6.26E−05 | 3.13E−04 | 1.3590 | 0.7738 | 1.25900 | 0.80370 |
| 11 | MET011 | 0.34 | 7.79E−05 | 3.22E−04 | 1.1830 | 1.2640 | 1.02300 | 1.18900 |
| 12 | MET012 | −0.28 | 1.23E−03 | 1.73E−03 | 1.1710 | 0.5940 | 1.14500 | 0.38850 |
| 13 | MET013 | 0.27 | 1.71E−03 | 2.19E−03 | 1.0310 | 1.3410 | 1.24400 | 2.83500 |
| 14 | MET014 | −0.33 | 1.13E−04 | 3.65E−04 | 1.2250 | 0.8626 | 1.12800 | 1.03600 |
| 15 | MET015 | 0.30 | 5.71E−04 | 1.03E−03 | 1.0450 | 0.4773 | 1.02400 | 0.61580 |
| 16 | MET016 | 0.34 | 9.66E−05 | 3.39E−04 | 1.1590 | 1.3540 | 0.96600 | 0.93210 |
| 17 | MET017 | −0.29 | 8.26E−04 | 1.38E−03 | 1.5890 | 3.4070 | 1.48300 | 2.02600 |
| 18 | MET018 | −0.33 | 1.45E−04 | 3.77E−04 | 1.0520 | 0.4583 | 1.11000 | 0.37320 |
| 19 | MET019 | −0.39 | 7.18E−06 | 7.79E−05 | 1.5600 | 1.4330 | 1.46900 | 0.86110 |
| 20 | MET020 | −0.33 | 1.28E−04 | 3.77E−04 | 1.0540 | 1.0130 | 1.10100 | 1.36800 |
| 21 | MET021 | 0.32 | 2.71E−04 | 5.53E−04 | 1.0910 | 0.2263 | 1.04500 | 0.17430 |
| 22 | MET022 | 0.30 | 6.09E−04 | 1.05E−03 | 1.0670 | 0.4767 | 1.01500 | 0.64190 |
| 23 | MET023 | −0.29 | 9.03E−04 | 1.45E−03 | 1.1910 | 0.6908 | 0.98500 | 0.45150 |
| 24 | MET024 | 0.27 | 2.10E−03 | 2.42E−03 | 1.2020 | 1.6930 | 1.17700 | 1.92700 |
| 25 | MET025 | 0.28 | 1.20E−03 | 1.73E−03 | 1.0830 | 0.3353 | 1.07500 | 0.42490 |
| 26 | MET026 | −0.36 | 3.36E−05 | 1.89E−04 | 1.1830 | 0.7660 | 1.25900 | 1.57400 |
| 27 | MET027 | 0.30 | 5.27E−04 | 9.87E−04 | 0.6510 | 1.3280 | 0.79710 | 2.76400 |
| 28 | MET028 | −0.34 | 7.87E−05 | 3.22E−04 | 2.3150 | 3.7920 | 2.15000 | 2.49800 |
| 29 | MET029 | −0.32 | 1.97E−04 | 4.43E−04 | 1.8980 | 3.4260 | 1.72500 | 1.99300 |
| 30 | MET030 | 0.29 | 1.11E−03 | 1.69E−03 | 0.3318 | 0.6587 | 0.04924 | 0.07526 |
| 31 | MET031 | −0.27 | 1.86E−03 | 2.26E−03 | 1.1650 | 0.3225 | 1.10400 | 0.44870 |
| 32 | MET032 | −0.28 | 1.13E−03 | 1.69E−03 | 1.2980 | 1.8830 | 1.00200 | 1.11000 |
| 33 | MET033 | 0.31 | 3.54E−04 | 6.93E−04 | 1.1460 | 0.7330 | 1.07300 | 0.72470 |
| 34 | MET034 | −0.32 | 1.92E−04 | 4.43E−04 | 1.3080 | 0.7954 | 2.12500 | 4.66500 |
| 35 | MET035 | −0.33 | 1.51E−04 | 3.77E−04 | 1.2400 | 0.7917 | 1.22700 | 0.32240 |
| 36 | MET036 | −0.22 | 1.28E−02 | 1.37E−02 | 1.2410 | 0.3775 | 1.23900 | 0.43790 |
| 37 | MET037 | 0.33 | 1.47E−04 | 3.77E−04 | 1.0040 | 0.2894 | 1.00600 | 0.46890 |
| 38 | MET038 | 0.27 | 2.21E−03 | 2.48E−03 | 0.9114 | 0.7129 | 0.92500 | 0.92550 |
| 39 | MET039 | 0.27 | 1.95E−03 | 2.30E−03 | 1.2390 | 1.5730 | 1.46000 | 1.58900 |
| 40 | MET040 | −0.28 | 1.46E−03 | 1.93E−03 | 1.2490 | 2.4680 | 0.73390 | 0.39890 |
| 41 | MET041 | −0.15 | 8.28E−02 | 8.28E−02 | 1.4410 | 0.9030 | 1.56500 | 1.31500 |
| 42 | MET042 | 0.23 | 8.84E−03 | 9.70E−03 | 0.9365 | 0.7594 | 0.97120 | 1.11200 |
| 43 | MET043 | 0.27 | 1.82E−03 | 2.26E−03 | 1.2770 | 0.8020 | 2.02600 | 5.76000 |
| 44 | MET044 | −0.19 | 2.95E−02 | 3.02E−02 | 1.0760 | 0.2946 | 1.01400 | 0.24710 |
| 45 | MET045 | 0.19 | 2.94E−02 | 3.02E−02 | 1.0830 | 0.6166 | 0.85060 | 0.41290 |

TABLE 4
Targeted Mass Spectrometry-Detected Blood Metabolites Associated
with Chronological Age in Modeling Cohort and Validation Cohort

| | | Correlation with | | | Relative Abundance | | Relative Abundance | |
|---|---|---|---|---|---|---|---|---|
| | | Chronological Age | | | in Modeling Cohort | | in Validation Cohort | |
| | Metabolite | Correlation | | | | Standard | | Standard |
| Importance | ID | Coefficient | p-Value | FDR | Mean | Deviation | Mean | Deviation |
| 1 | MET001 | −0.21 | 5.43E−05 | 1.10E−04 | 1.8610 | 1.3230 | 1.5420 | 1.0500 |
| 2 | MET002 | −0.18 | 3.33E−04 | 5.18E−04 | 1.2440 | 0.7932 | 1.0650 | 0.7348 |
| 3 | MET003 | −0.23 | 6.97E−06 | 2.06E−05 | 1.4680 | 0.9409 | 1.2900 | 0.8468 |
| 4 | MET004 | −0.17 | 1.06E−03 | 1.43E−03 | 1.0660 | 0.6645 | 0.9139 | 0.5191 |
| 5 | MET005 | −0.35 | 2.98E−12 | 1.95E−11 | 1.9560 | 1.2810 | 1.6030 | 0.9889 |
| 6 | MET006 | 0.08 | 1.03E−01 | 1.05E−01 | 1.1750 | 0.4797 | 1.0630 | 0.3842 |
| 7 | MET007 | 0.22 | 2.45E−05 | 5.55E−05 | 1.3070 | 0.6572 | 1.2580 | 0.5505 |
| 8 | MET008 | −0.45 | 1.92E−20 | 3.78E−19 | 1.6890 | 0.9418 | 1.5440 | 0.9484 |
| 9 | MET009 | 0.12 | 1.65E−02 | 1.73E−02 | 1.3660 | 4.8450 | 0.9704 | 0.7540 |
| 10 | MET010 | 0.11 | 2.76E−02 | 2.86E−02 | 0.3379 | 0.6533 | 0.3171 | 0.2969 |
| 11 | MET011 | 0.13 | 9.73E−03 | 1.04E−02 | 1.1080 | 1.6970 | 1.0760 | 1.0540 |
| 12 | MET012 | −0.19 | 2.92E−04 | 4.66E−04 | 0.8342 | 0.7169 | 0.8258 | 0.5818 |
| 13 | MET013 | 0.07 | 1.59E−01 | 1.59E−01 | 1.9220 | 3.5720 | 1.3880 | 1.9700 |
| 14 | MET014 | −0.38 | 2.17E−14 | 1.83E−13 | 1.5100 | 1.1290 | 1.4620 | 1.3400 |
| 15 | MET015 | 0.27 | 1.26E−07 | 5.31E−07 | 1.0500 | 0.4696 | 1.0510 | 0.4744 |
| 16 | MET046 | −0.47 | 2.23E−22 | 6.58E−21 | 0.8888 | 0.4034 | 0.8031 | 0.4267 |
| 17 | MET019 | −0.48 | 8.35E−23 | 4.93E−21 | 1.4150 | 0.6549 | 1.3060 | 0.6637 |
| 18 | MET047 | −0.41 | 8.56E−17 | 1.26E−15 | 1.9290 | 4.3680 | 1.4090 | 2.5400 |
| 19 | MET048 | 0.27 | 6.60E−08 | 3.24E−07 | 1.5610 | 1.1370 | 1.4920 | 1.0300 |
| 20 | MET049 | 0.25 | 9.56E−07 | 3.32E−06 | 1.0760 | 0.6407 | 0.9764 | 0.4863 |
| 21 | MET050 | 0.26 | 1.94E−07 | 7.62E−07 | 1.0520 | 0.7390 | 1.0130 | 0.6448 |
| 22 | MET045 | 0.22 | 1.79E−05 | 4.39E−05 | 1.1620 | 0.6130 | 1.1580 | 0.6634 |
| 23 | MET051 | 0.26 | 2.52E−07 | 9.28E−07 | 1.4180 | 1.1170 | 1.3450 | 1.0150 |
| 24 | MET052 | 0.19 | 2.01E−04 | 3.39E−04 | 1.1020 | 0.7191 | 1.0140 | 0.6439 |
| 25 | MET053 | −0.40 | 4.09E−16 | 4.82E−15 | 0.9806 | 0.7149 | 0.9439 | 0.8545 |
| 26 | MET054 | −0.39 | 1.06E−14 | 1.04E−13 | 1.3340 | 0.6198 | 1.2520 | 0.6447 |
| 27 | MET055 | 0.17 | 1.18E−03 | 1.54E−03 | 0.1388 | 0.2178 | 0.1997 | 0.3362 |
| 28 | MET056 | 0.15 | 3.51E−03 | 3.84E−03 | 1.1050 | 0.4817 | 1.0760 | 0.4139 |
| 29 | MET057 | 0.15 | 2.86E−03 | 3.18E−03 | 5.5960 | 11.8300 | 5.5270 | 10.7900 |
| 30 | MET058 | 0.19 | 2.74E−04 | 4.48E−04 | 1.0620 | 0.5994 | 0.9624 | 0.4871 |
| 31 | MET059 | 0.20 | 8.75E−05 | 1.67E−04 | 1.0230 | 1.8190 | 0.7890 | 0.8641 |
| 32 | MET032 | −0.24 | 2.91E−06 | 9.03E−06 | 3.3640 | 5.6180 | 2.1100 | 2.8140 |
| 33 | MET024 | 0.17 | 8.26E−04 | 1.13E−03 | 0.6594 | 0.7967 | 0.6186 | 0.9501 |
| 34 | MET038 | 0.22 | 1.19E−05 | 3.18E−05 | 1.0380 | 0.3750 | 1.0500 | 0.3849 |
| 35 | MET060 | 0.20 | 1.24E−04 | 2.28E−04 | 1.0620 | 2.0000 | 0.8072 | 0.9094 |
| 36 | MET061 | 0.16 | 1.78E−03 | 2.10E−03 | 0.2462 | 0.3100 | 0.2750 | 0.3920 |
| 37 | MET062 | 0.15 | 2.86E−03 | 3.18E−03 | 1.3300 | 0.8940 | 1.1740 | 0.7123 |
| 38 | MET018 | −0.19 | 1.65E−04 | 2.94E−04 | 1.8600 | 0.6453 | 1.8030 | 0.5706 |
| 39 | MET017 | −0.24 | 2.07E−06 | 6.78E−06 | 3.6860 | 6.1050 | 2.1320 | 2.8270 |
| 40 | MET063 | −0.23 | 1.08E−05 | 3.03E−05 | 2.2060 | 3.3100 | 1.6630 | 2.7460 |
| 41 | MET064 | 0.18 | 3.95E−04 | 5.98E−04 | 0.3381 | 0.3485 | 0.3467 | 0.3014 |
| 42 | MET065 | 0.18 | 5.77E−04 | 8.31E−04 | 1.2210 | 0.3468 | 1.2240 | 0.3272 |
| 43 | MET066 | −0.20 | 6.83E−05 | 1.34E−04 | 2.5610 | 3.2120 | 2.3160 | 4.1360 |
| 44 | MET067 | −0.18 | 6.59E−04 | 9.25E−04 | 2.9990 | 4.9060 | 2.4620 | 4.4130 |
| 45 | MET068 | 0.16 | 1.37E−03 | 1.71E−03 | 0.9324 | 1.1870 | 0.7504 | 0.8968 |
| 46 | MET042 | 0.16 | 1.42E−03 | 1.75E−03 | 0.8080 | 1.1900 | 0.6301 | 0.5678 |
| 47 | MET069 | −0.18 | 4.53E−04 | 6.69E−04 | 4.2060 | 8.7120 | 3.2070 | 5.2500 |
| 48 | MET023 | −0.36 | 6.02E−13 | 4.44E−12 | 1.4210 | 0.8907 | 1.2410 | 0.7257 |
| 49 | MET070 | −0.22 | 1.40E−05 | 3.59E−05 | 3.1070 | 5.4560 | 2.6450 | 5.0360 |
| 50 | MET071 | 0.16 | 1.72E−03 | 2.07E−03 | 1.5130 | 0.7199 | 1.4050 | 0.7886 |
| 51 | MET072 | −0.28 | 3.10E−08 | 1.83E−07 | 1.3720 | 0.7852 | 1.2590 | 0.8169 |
| 52 | MET073 | 0.17 | 1.33E−03 | 1.70E−03 | 0.8898 | 1.5350 | 0.6814 | 1.3260 |
| 53 | MET074 | −0.21 | 3.14E−05 | 6.86E−05 | 2.9840 | 5.5130 | 2.5870 | 5.6530 |
| 54 | MET075 | −0.22 | 1.88E−05 | 4.44E−05 | 1.3120 | 0.7783 | 1.1800 | 0.6807 |
| 55 | MET076 | 0.16 | 2.06E−03 | 2.39E−03 | 1.3170 | 1.7390 | 1.0360 | 0.8784 |
| 56 | MET020 | −0.19 | 1.92E−04 | 3.33E−04 | 1.6030 | 1.1000 | 1.3590 | 0.9732 |
| 57 | MET077 | −0.27 | 7.10E−08 | 3.24E−07 | 1.1040 | 0.8141 | 0.9301 | 0.6233 |
| 58 | MET040 | −0.28 | 4.90E−08 | 2.62E−07 | 1.3870 | 0.7935 | 1.2650 | 0.8124 |
| 59 | MET026 | −0.21 | 5.30E−05 | 1.10E−04 | 1.7110 | 1.4700 | 1.4980 | 1.2970 |

### Embodiment 3 Model for Predicting Physiological Age Using Metabolite Biomarkers and Validation

Using the random forest approach, the importance of 45 high-resolution-detected metabolites and 59 targeted-detected metabolites was ranked respectively. Starting with the 15 core metabolites, the model's predictive performance was continuously explored through incremental expansion.

A linear regression model was constructed using blood metabolites screened through correlation analysis. This model is capable of utilizing these blood metabolites to predict an individual's physiological age, evaluated based on RMSE, MAE, R-squared, and correlation coefficient (R).

1. Physiological Age Prediction Performance Based on High-Resolution Mass Spectrometry Blood Metabolites.

TABLE 5
Physiological Age Prediction Performance of 15 Core Metabolites

| | Evaluation | Modeling | Validation |
|---|---|---|---|
| Model Metabolite Combination1 | Metrics | Cohort | Cohort |
| MET001, MET002, MET003, | RMSE | 5.58 | 6.33 |
| MET004, MET005, MET006, | MAE | 4.36 | 5.35 |
| MET007, MET008, MET009, | R-squared | 0.49 | 0.38 |
| MET010, MET011, MET012, | Correlation | 0.7 | 0.61 |
| MET013, MET014, MET015 | Coefficient | | |
| | (R) | | |

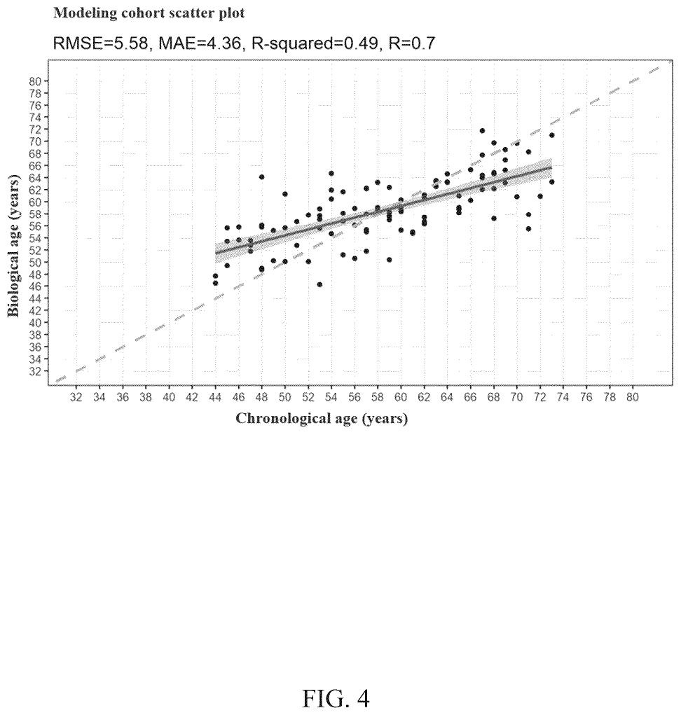

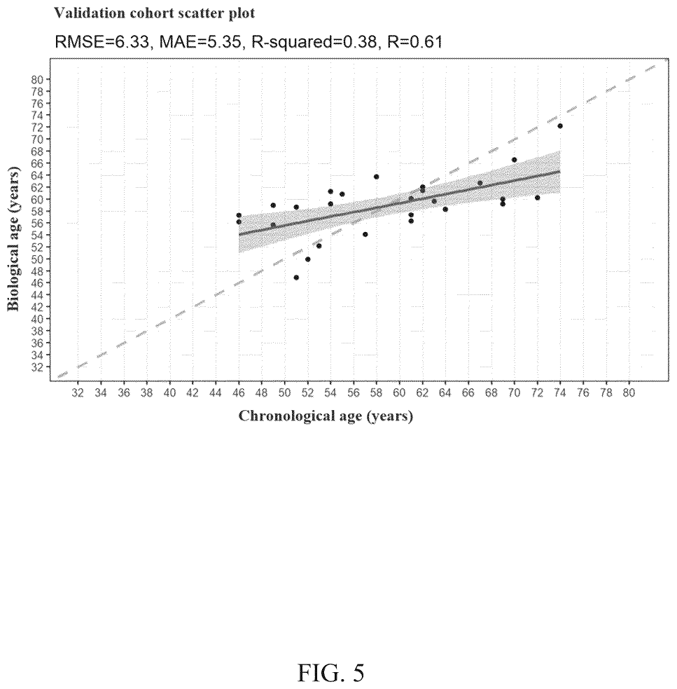

FIG. 4 is a scatter plot of the modeling cohort prediction results in the physiological age prediction performance of the 15 core metabolites based on high-resolution mass spectrometry-based metabolomics detection, and FIG. 5 is a scatter plot of the validation cohort prediction results in the physiological age prediction performance of the 15 core metabolites based on high-resolution mass spectrometry-based metabolomics detection. Where the x-axis represents chronological age, and the y-axis represents predicted physiological age.

TABLE 6
Physiological Age Prediction Performance of 45 Blood Metabolites
All Strongly Correlated with Chronological Age

| | Evaluation | Modeling | Validation |
|---|---|---|---|
| Model Metabolite Combination2 | Metrics | Cohort | Cohort |
| MET001, MET002, MET003, | RMSE | 4.26 | 4.83 |
| MET004, MET005, MET006, | MAE | 3.28 | 3.81 |
| MET007, MET008, MET009, | R-squared | 0.7 | 0.65 |
| MET010, MET011, MET012, | Correlation | 0.84 | 0.81 |
| MET013, MET014, MET015, | Coefficient | | |
| MET016, MET017, MET018, | (R) | | |
| MET019, MET020, MET021, | | | |
| MET022, MET023, MET024, | | | |
| MET025, MET026, MET027, | | | |
| MET028, MET029, MET030, | | | |
| MET031, MET032, MET033, | | | |
| MET034, MET035, MET036, | | | |
| MET037, MET038, MET039, | | | |
| MET040, MET041, MET042, | | | |
| MET043, MET044, MET045 | | | |

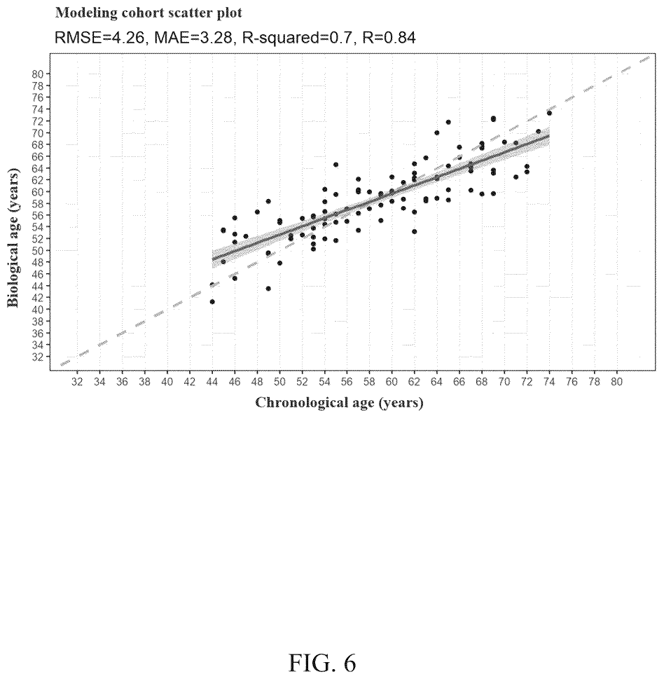

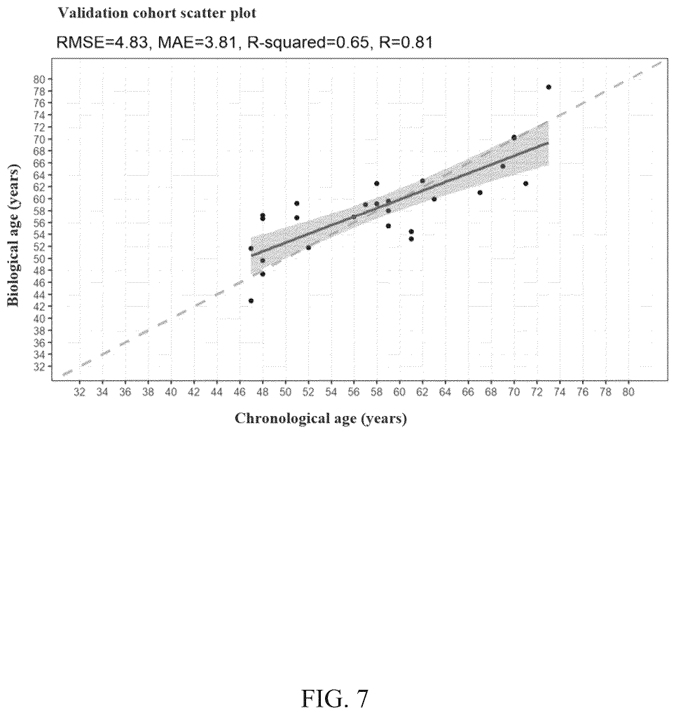

FIG. 6 is a scatter plot of the modeling cohort prediction results in the physiological age prediction performance of the 45 blood metabolites all strongly correlated with chronological age based on high-resolution mass spectrometry-based metabolomics detection, and FIG. 7 is a scatter plot of the validation cohort prediction results in the physiological age prediction performance of the 45 blood metabolites all strongly correlated with chronological age based on high-resolution mass spectrometry-based metabolomics detection. Where the x-axis represents chronological age, and the y-axis represents predicted physiological age.

TABLE 7
Physiological Age Prediction Performance of Adding Several Blood Metabolites
Strongly Correlated with Chronological Age to the 15 Core Metabolites

| | Evaluation | Modeling | Validation |
|---|---|---|---|
| | Metrics | Cohort | Cohort |
| Model Metabolite Combination3 | | | |
| MET001, MET002, MET003, MET004, MET005, | RMSE | 5.62 | 5.84 |
| MET006, MET007, MET008, MET009, MET010, | MAE | 4.46 | 4.74 |
| MET011, MET012, MET013, MET014, MET015, | R-squared | 0.5 | 0.42 |
| MET016, MET017, MET018, MET019, MET020 | Correlation | 0.7 | 0.65 |
| | Coefficient | | |
| | (R) | | |
| Model Metabolite Combination4 | | | |
| MET001, MET002, MET003, MET004, MET005, | RMSE | 5.08 | 5.83 |
| MET006, MET007, MET008, MET009, MET010, | MAE | 3.97 | 4.81 |
| MET011, MET012, MET013, MET014, MET015, | R-squared | 0.58 | 0.49 |
| MET016, MET017, MET018, MET019, MET020, | Correlation | 0.76 | 0.7 |
| MET021, MET022, MET023, MET024, MET025 | Coefficient | | |
| | (R) | | |
| Model Metabolite Combination5 | | | |
| MET001, MET002, MET003, MET004, MET005, | RMSE | 4.89 | 4.32 |
| MET006, MET007, MET008, MET009, MET010, | MAE | 3.89 | 3.17 |
| MET011, MET012, MET013, MET014, MET015, | R-squared | 0.64 | 0.57 |
| MET016, MET017, MET018, MET019, MET020, | Correlation | 0.8 | 0.78 |
| MET021, MET022, MET023, MET024, MET025, | Coefficient | | |
| MET026, MET027, MET028, MET029, MET030 | (R) | | |
| Model Metabolite Combination6 | | | |
| MET001, MET002, MET003, MET004, MET005, | RMSE | 4.76 | 4.45 |
| MET006, MET007, MET008, MET009, MET010, | MAE | 3.81 | 3.59 |
| MET011, MET012, MET013, MET014, MET015, | R-squared | 0.65 | 0.59 |
| MET016, MET017, MET018, MET019, MET020, | Correlation | 0.81 | 0.84 |
| MET021, MET022, MET023, MET024, MET025, | Coefficient | | |
| MET026, MET027, MET028, MET029, MET030, | (R) | | |
| MET031, MET032, MET033, MET034, MET035 | | | |
| Model Metabolite Combination7 | | | |
| MET001, MET002, MET003, MET004, MET005, | RMSE | 4.45 | 4.24 |
| MET006, MET007, MET008, MET009, MET010, | MAE | 3.47 | 3.31 |
| MET011, MET012, MET013, MET014, MET015, | R-squared | 0.69 | 0.64 |
| MET016, MET017, MET018, MET019, MET020, | Correlation | 0.83 | 0.82 |
| MET021, MET022, MET023, MET024, MET025, | Coefficient | | |
| MET026, MET027, MET028, MET029, MET030, | (R) | | |
| MET031, MET032, MET033, MET034, MET035, | | | |
| MET036, MET037, MET038, MET039, MET040 | | | |

2. Physiological Age Prediction Performance of Targeted Mass Spectrometry-Based Blood Metabolites.

TABLE 8
Physiological Age Prediction Performance of 15 Core Metabolites

| | Evaluation | Modeling | Validation |
|---|---|---|---|
| Model Metabolite Combination1 | Metrics | Cohort | Cohort |
| MET001, MET002, MET003, | RMSE | 7.32 | 7.75 |
| MET004, MET005, MET006, | MAE | 5.94 | 6.44 |
| MET007, MET008, MET009, | R-squared | 0.37 | 0.34 |
| MET010, MET011, MET012, | Correlation | 0.61 | 0.58 |
| MET013, MET014, MET015 | Coefficient | | |
| | (R) | | |

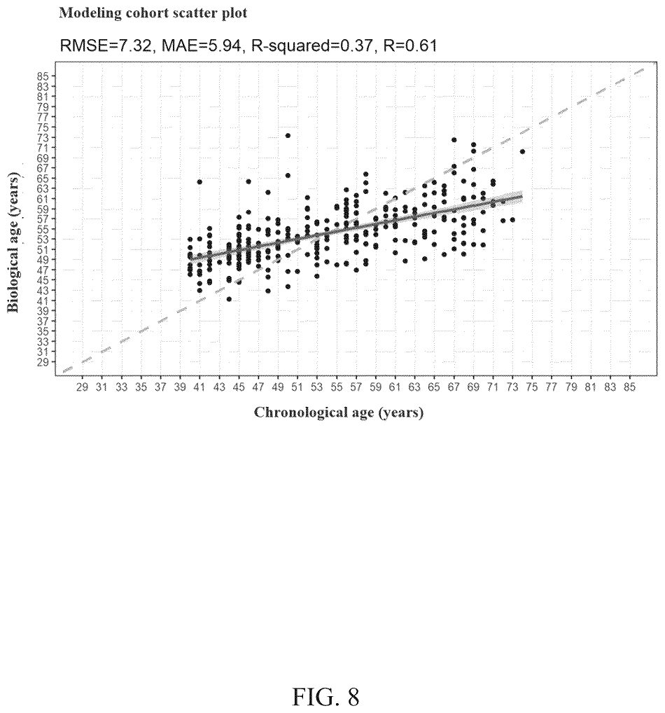

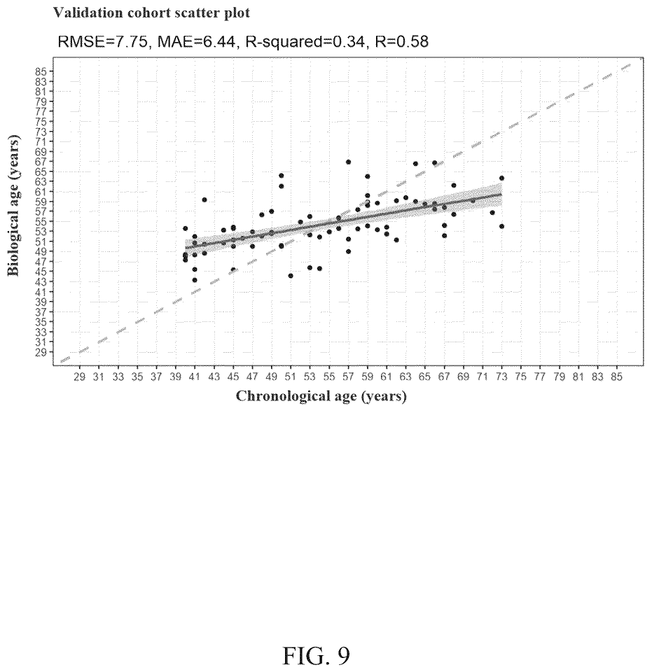

FIG. 8 is a scatter plot of the modeling cohort prediction results in the physiological age prediction performance of the 15 core metabolites based on targeted mass spectrometry-based metabolomics detection, and FIG. 9 is a scatter plot of the validation cohort prediction results in the physiological age prediction performance of the 15 core metabolites based on targeted mass spectrometry-based metabolomics detection. Where the x-axis represents chronological age, and the y-axis represents predicted physiological age.

TABLE 9
Physiological Age Prediction Performance of 59 Blood Metabolites
All Strongly Correlated with Chronological Age

| | Evaluation | Modeling | Validation |
|---|---|---|---|
| Model Metabolite Combination2 | Metrics | Cohort | Cohort |
| MET001, MET002, MET003, | RMSE | 5.85 | 5.65 |
| MET004, MET005, MET006, | MAE | 4.58 | 4.63 |
| MET007, MET008, MET009, | R-squared | 0.62 | 0.55 |
| MET010, MET011, MET012, | Correlation | 0.79 | 0.74 |
| MET013, MET014, MET015, | Coefficient | | |
| MET046, MET019, MET047, | (R) | | |
| MET048, MET049, MET050, | | | |
| MET045, MET051, MET052, | | | |
| MET053, MET054, MET055, | | | |
| MET056, MET057, MET058, | | | |
| MET059, MET032, MET024, | | | |
| MET038, MET060, MET061, | | | |
| MET062, MET018, MET017, | | | |
| MET063, MET064, MET065, | | | |
| MET066, MET067, MET068, | | | |
| MET042, MET069, MET023, | | | |
| MET070, MET071, MET072, | | | |
| MET073, MET074, MET075, | | | |
| MET076, MET020, MET077, | | | |
| MET040, MET026 | | | |

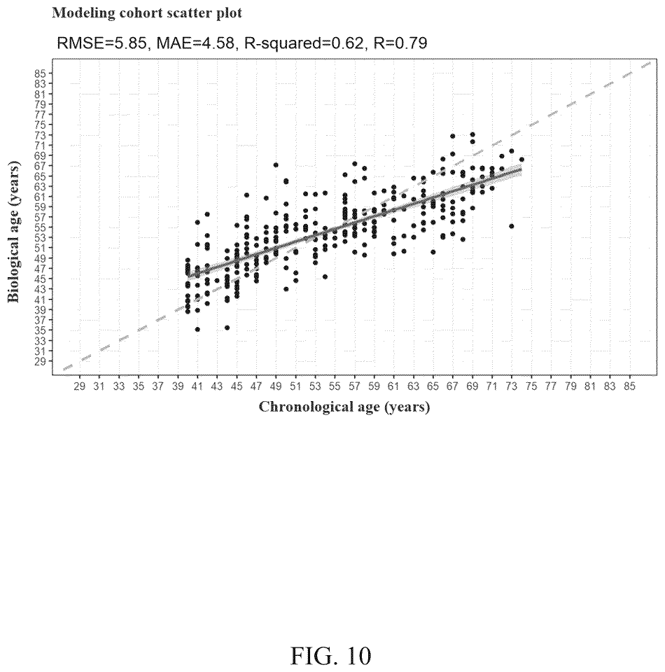

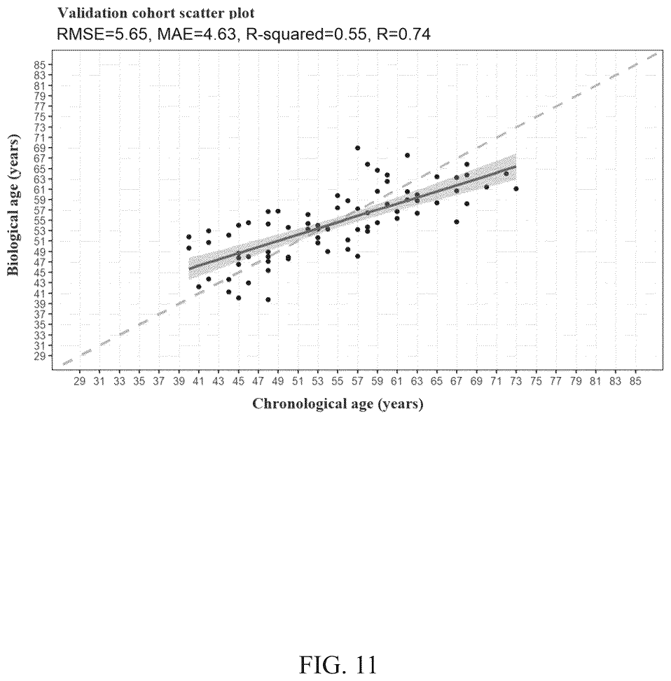

FIG. 10 is a scatter plot of the modeling cohort prediction results in the physiological age prediction performance of the 59 blood metabolites all strongly correlated with chronological age based on targeted mass spectrometry-based metabolomics detection, and FIG. 11 is a scatter plot of the validation cohort prediction results in the physiological age prediction performance of the 59 blood metabolites all strongly correlated with chronological age based on targeted mass spectrometry-based metabolomics detection. Where the x-axis represents chronological age, and the y-axis represents predicted physiological age.

TABLE 10
Physiological Age Prediction Performance of Adding Several Blood Metabolites
Strongly Correlated with Chronological Age to the 15 Core Metabolites

| | Evaluation | Modeling | Validation |
|---|---|---|---|
| | Metrics | Cohort | Cohort |
| Model Metabolite Combination3 | | | |
| MET001, MET002, MET003, MET004, MET005, | RMSE | 6.67 | 7.27 |
| MET006, MET007, MET008, MET009, MET010, | MAE | 5.33 | 6 |
| MET011, MET012, MET013, MET014, MET015, | R-squared | 0.48 | 0.4 |
| MET046, MET019, MET047, MET048, MET049 | Correlation | 0.69 | 0.65 |
| | Coefficient | | |
| | (R) | | |
| Model Metabolite Combination4 | | | |
| MET001, MET002, MET003, MET004, MET005, | RMSE | 6.38 | 7.44 |
| MET006, MET007, MET008, MET009, MET010, | MAE | 5.19 | 5.92 |
| MET011, MET012, MET013, MET014, MET015, | R-squared | 0.51 | 0.43 |
| MET046, MET019, MET047, MET048, MET049, | Correlation | 0.71 | 0.68 |
| MET050, MET045, MET051, MET052, MET053 | Coefficient | | |
| | (R) | | |
| Model Metabolite Combination5 | | | |
| MET001, MET002, MET003, MET004, MET005, | RMSE | 6.24 | 7.01 |
| MET006, MET007, MET008, MET009, MET010, | MAE | 5.01 | 5.66 |
| MET011, MET012, MET013, MET014, MET015, | R-squared | 0.54 | 0.47 |
| MET046, MET019, MET047, MET048, MET049, | Correlation | 0.73 | 0.69 |
| MET050, MET045, MET051, MET052, MET053, | Coefficient | | |
| MET054, MET055, MET056, MET057, MET058 | (R) | | |
| Model Metabolite Combination6 | | | |
| MET001, MET002, MET003, MET004, MET005, | RMSE | 6.12 | 6.51 |
| MET006, MET007, MET008, MET009, MET010, | MAE | 4.87 | 5.24 |
| MET011, MET012, MET013, MET014, MET015, | R-squared | 0.57 | 0.49 |
| MET046, MET019, MET047, MET048, MET049, | Correlation | 0.75 | 0.7 |
| MET050, MET045, MET051, MET052, MET053, | Coefficient | | |
| MET054, MET055, MET056, MET057, MET058, | (R) | | |
| MET059, MET032, MET024, MET038, MET060 | | | |
| Model Metabolite Combination7 | | | |
| MET001, MET002, MET003, MET004, MET005, | RMSE | 6 | 6.47 |
| MET006, MET007, MET008, MET009, MET010, | MAE | 4.82 | 5.05 |
| MET011, MET012, MET013, MET014, MET015, | R-squared | 0.58 | 0.52 |
| MET046, MET019, MET047, MET048, MET049, | Correlation | 0.76 | 0.73 |
| MET050, MET045, MET051, MET052, MET053, | Coefficient | | |
| MET054, MET055, MET056, MET057, MET058, | (R) | | |
| MET059, MET032, MET024, MET038, MET060, | | | |
| MET061, MET062, MET018, MET017, MET063 | | | |
| Model Metabolite Combination8 | | | |
| MET001, MET002, MET003, MET004, MET005, | RMSE | 6.01 | 6.05 |
| MET006, MET007, MET008, MET009, MET010, | MAE | 4.8 | 4.86 |
| MET011, MET012, MET013, MET014, MET015, | R-squared | 0.59 | 0.53 |
| MET046, MET019, MET047, MET048, MET049, | Correlation | 0.77 | 0.73 |
| MET050, MET045, MET051, MET052, MET053, | Coefficient | | |
| MET054, MET055, MET056, MET057, MET058, | (R) | | |
| MET059, MET032, MET024, MET038, MET060, | | | |
| MET061, MET062, MET018, MET017, MET063, | | | |
| MET064, MET065, MET066, MET067, MET068 | | | |
| Model Metabolite Combination9 | | | |
| MET001, MET002, MET003, MET004, MET005, | RMSE | 5.83 | 6.63 |
| MET006, MET007, MET008, MET009, MET010, | MAE | 4.65 | 5.34 |
| MET011, MET012, MET013, MET014, MET015, | R-squared | 0.6 | 0.52 |
| MET046, MET019, MET047, MET048, MET049, | Correlation | 0.77 | 0.72 |
| MET050, MET045, MET051, MET052, MET053, | Coefficient | | |
| MET054, MET055, MET056, MET057, MET058, | (R) | | |
| MET059, MET032, MET024, MET038, MET060, | | | |
| MET061, MET062, MET018, MET017, MET063, | | | |
| MET064, MET065, MET066, MET067, MET068, | | | |
| MET042, MET069, MET023, MET070, MET071 | | | |
| Model Metabolite Combination10 | | | |
| MET001, MET002, MET003, MET004, MET005, | RMSE | 5.72 | 6.24 |
| MET006, MET007, MET008, MET009, MET010, | MAE | 4.59 | 4.6 |
| MET011, MET012, MET013, MET014, MET015, | R-squared | 0.62 | 0.55 |
| MET046, MET019, MET047, MET048, MET049, | Correlation | 0.78 | 0.74 |
| MET050, MET045, MET051, MET052, MET053, | Coefficient | | |
| MET054, MET055, MET056, MET057, MET058, | (R) | | |
| MET059, MET032, MET024, MET038, MET060, | | | |
| MET061, MET062, MET018, MET017, MET063, | | | |
| MET064, MET065, MET066, MET067, MET068, | | | |
| MET042, MET069, MET023, MET070, MET071, | | | |
| MET072, MET073, MET074, MET075, MET076 | | | |

### Embodiment 4 Validation of Tumor-Induced Accelerated Aging

Healthy individuals and colorectal cancer patients of the same chronological age group were selected, and their physiological ages were predicted through the aforementioned established high-resolution mass spectrometry detection and targeted mass spectrometry detection metabolomics blood metabolite models, respectively.

TABLE 11
Age Distribution of Colorectal Tumor Cohort

| | | Age Distribution | | | | |
|---|---|---|---|---|---|---|
| | | | Mean ± Standard | | | |
| | | Number | Deviation | Minimum | Median | Maximum |
| High-Resolution | Total | 206 | 59.67 ± 8.57 | 32 | 60 | 81 |
| Detection Colorectal | Male | 131 | 59.1 ± 8.39 | 32 | 59 | 78 |
| Tumor Cohort | Female | 75 | 60.67 ± 8.84 | 40 | 62 | 81 |
| Targeted Detection | Total | 173 | 58.35 ± 10.09 | 32 | 59 | 85 |
| Colorectal Tumor | Male | 112 | 58.74 ± 9.88 | 32 | 59 | 78 |
| Cohort | Female | 61 | 57.64 ± 10.51 | 41 | 59 | 85 |

TABLE 12
High-Resolution Mass Spectrometry-Detected Blood
Metabolites in Colorectal Tumor Cohort

| | |
|---|---|
| | Relative Abundance in |
| Metabolite | Colorectal Tumor Cohort |

| Importance | ID | Mean | Standard Deviation |
|---|---|---|---|
| 1 | MET001 | 1.081 | 0.6693 |
| 2 | MET002 | 1.089 | 0.699 |
| 3 | MET003 | 1.123 | 0.7304 |
| 4 | MET004 | 1.153 | 0.822 |
| 5 | MET005 | 1.099 | 0.5554 |
| 6 | MET006 | 1.443 | 0.6691 |
| 7 | MET007 | 0.8972 | 0.4676 |
| 8 | MET008 | 1.096 | 0.768 |
| 9 | MET009 | 1.318 | 1.362 |
| 10 | MET010 | 1.493 | 1.048 |
| 11 | MET011 | 1.044 | 1.124 |
| 12 | MET012 | 1.111 | 0.5092 |
| 13 | MET013 | 1.135 | 2.872 |
| 14 | MET014 | 1.159 | 0.9585 |
| 15 | MET015 | 0.9086 | 0.5282 |
| 16 | MET016 | 1.05 | 1.233 |
| 17 | MET017 | 1.154 | 2.233 |
| 18 | MET018 | 0.8565 | 0.3592 |
| 19 | MET019 | 1.311 | 0.6993 |
| 20 | MET020 | 1.204 | 1.231 |
| 21 | MET021 | 1.15 | 0.2507 |
| 22 | MET022 | 1.321 | 1.21 |
| 23 | MET023 | 1.313 | 1.131 |
| 24 | MET024 | 1.281 | 1.975 |
| 25 | MET025 | 1.284 | 2.237 |
| 26 | MET026 | 1.183 | 0.7161 |
| 27 | MET027 | 0.7314 | 1.445 |
| 28 | MET028 | 2.058 | 2.018 |
| 29 | MET029 | 1.485 | 2.582 |
| 30 | MET030 | 1.382 | 14.43 |
| 31 | MET031 | 1.144 | 0.721 |
| 32 | MET032 | 0.9536 | 1.474 |
| 33 | MET033 | 1.082 | 0.7397 |
| 34 | MET034 | 0.9641 | 0.6502 |
| 35 | MET035 | 1.22 | 0.7709 |
| 36 | MET036 | 1.189 | 0.3287 |
| 37 | MET037 | 1.048 | 0.8482 |
| 38 | MET038 | 0.9747 | 0.7695 |
| 39 | MET039 | 1.212 | 1.104 |
| 40 | MET040 | 1.049 | 1.514 |
| 41 | MET041 | 1.117 | 0.6282 |
| 42 | MET042 | 1.445 | 2.32 |
| 43 | MET043 | 1.118 | 1.235 |
| 44 | MET044 | 1.034 | 0.2556 |
| 45 | MET045 | 1.131 | 0.5074 |

TABLE 13
Targeted Mass Spectrometry Detection of Blood
Metabolites in Colorectal Tumor Cohort

| | |
|---|---|
| | Relative Abundance in |
| Metabolite | Colorectal Tumor Cohort |

| Importance | ID | Mean | Standard Deviation |
|---|---|---|---|
| 1 | MET001 | 1.332 | 0.9421 |
| 2 | MET002 | 1.184 | 0.7875 |
| 3 | MET003 | 1.117 | 0.7486 |
| 4 | MET004 | 0.9695 | 0.6444 |
| 5 | MET005 | 1.464 | 0.86 |
| 6 | MET006 | 1.033 | 0.4109 |
| 7 | MET007 | 1.11 | 0.6017 |
| 8 | MET008 | 1.311 | 0.7605 |
| 9 | MET009 | 2.895 | 13.46 |
| 10 | MET010 | 0.7695 | 4.942 |
| 11 | MET011 | 1.244 | 1.472 |
| 12 | MET012 | 0.783 | 1.397 |
| 13 | MET013 | 1.17 | 2.565 |
| 14 | MET014 | 1.18 | 0.7529 |
| 15 | MET015 | 0.9353 | 0.4677 |
| 16 | MET046 | 0.7952 | 0.432 |
| 17 | MET019 | 1.165 | 0.578 |
| 18 | MET047 | 1.202 | 2.324 |
| 19 | MET048 | 1.177 | 0.8676 |
| 20 | MET049 | 1.164 | 0.6315 |
| 21 | MET050 | 0.8204 | 0.6249 |
| 22 | MET045 | 1.303 | 0.7254 |
| 23 | MET051 | 1.18 | 1.017 |
| 24 | MET052 | 1.237 | 0.7232 |
| 25 | MET053 | 0.8519 | 0.5799 |
| 26 | MET054 | 1.107 | 0.4844 |
| 27 | MET055 | 0.4025 | 0.4433 |
| 28 | MET056 | 1.205 | 0.5227 |
| 29 | MET057 | 8.869 | 21.9 |
| 30 | MET058 | 1.194 | 0.6895 |
| 31 | MET059 | 0.9458 | 1.768 |
| 32 | MET032 | 1.822 | 2.79 |
| 33 | MET024 | 0.607 | 0.9153 |
| 34 | MET038 | 1.106 | 0.4068 |
| 35 | MET060 | 0.9383 | 1.867 |
| 36 | MET061 | 0.4873 | 0.4517 |
| 37 | MET062 | 1.112 | 0.796 |
| 38 | MET018 | 1.586 | 0.4572 |
| 39 | MET017 | 2.114 | 4.251 |
| 40 | MET063 | 1.328 | 1.197 |
| 41 | MET064 | 0.5882 | 0.5986 |
| 42 | MET065 | 1.073 | 0.3883 |
| 43 | MET066 | 1.421 | 1.176 |
| 44 | MET067 | 1.712 | 3.514 |
| 45 | MET068 | 0.8167 | 1.193 |
| 46 | MET042 | 1.037 | 1.476 |
| 47 | MET069 | 2.42 | 5.147 |
| 48 | MET023 | 1.243 | 0.6393 |
| 49 | MET070 | 1.965 | 3.856 |
| 50 | MET071 | 1.33 | 0.5891 |
| 51 | MET072 | 1.276 | 0.7515 |
| 52 | MET073 | 0.6896 | 1.329 |
| 53 | MET074 | 2.051 | 4.859 |
| 54 | MET075 | 1.023 | 0.5861 |
| 55 | MET076 | 1.424 | 1.909 |
| 56 | MET020 | 1.299 | 0.9471 |
| 57 | MET077 | 1.001 | 0.6451 |
| 58 | MET040 | 1.28 | 0.7755 |
| 59 | MET026 | 1.235 | 0.9709 |

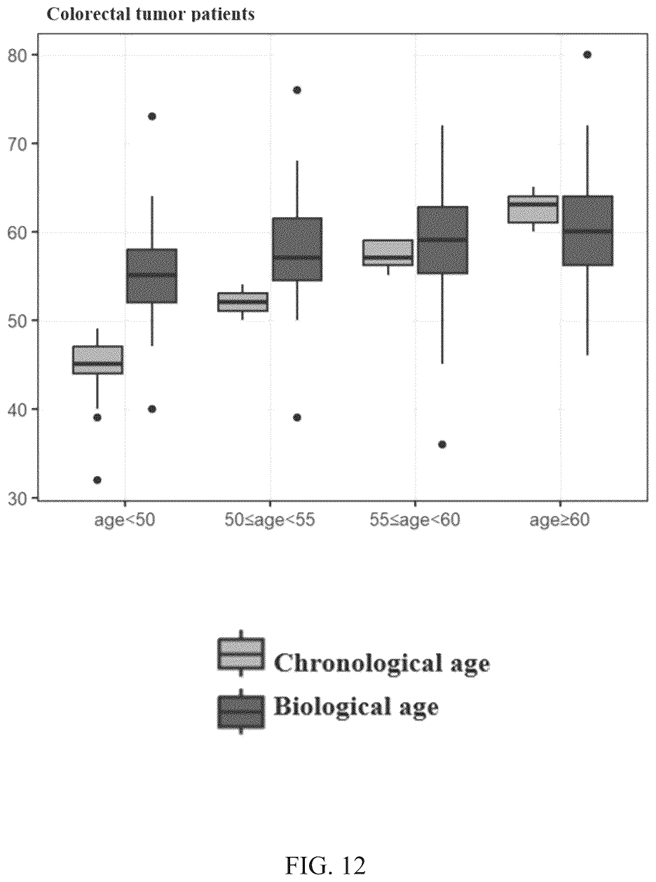

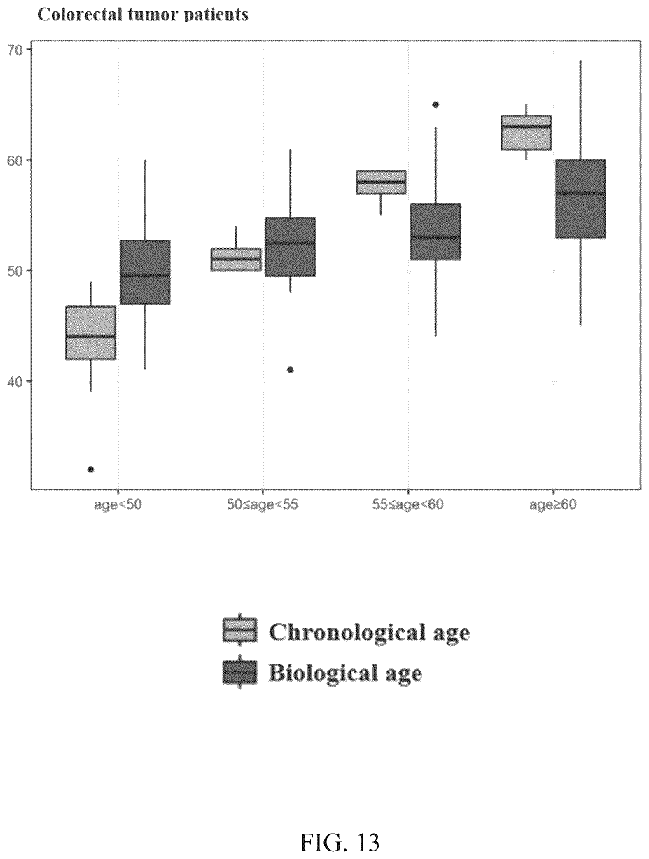

The performance of the high-resolution mass spectrometry detection metabolite model in colorectal tumor patients is shown in FIG. 12, and the performance of the targeted mass spectrometry detection metabolite model in colorectal tumor patients is shown in FIG. 13. In the same calendar age groups, the physiological age predicted by blood metabolites in the colorectal tumor group, particularly in patients under 45 years, was significantly higher than that in the healthy individual group. This trend demonstrates that predictive models using blood metabolite biomarkers can effectively identify accelerated aging patterns induced by colorectal tumors.

Having described the basic concepts herein, it is apparent to those skilled in the art that the foregoing detailed disclosure is merely exemplary in nature and does not constitute a limitation to the present disclosure. Although not explicitly stated herein, those skilled in the art may make various modifications, improvements, and amendments to the present disclosure. Such modifications, improvements, and amendments are suggested in the present disclosure and therefore remain within the spirit and scope of the exemplary embodiments of the present disclosure.

Furthermore, the present disclosure uses specific terminology to describe the embodiments of the present disclosure. Terms such as “an embodiment,” “one embodiment,” and/or “some embodiments” refer to a specific feature, structure, or characteristic associated with at least one embodiment of the present disclosure. Therefore, it should be emphasized and noted that references to “an embodiment,” “one embodiment,” or “an alternative embodiment” made twice or more in different parts of the present disclosure do not necessarily refer to the same embodiment. Additionally, certain features, structures, or characteristics in one or more embodiments of the present disclosure may be appropriately combined.

Moreover, unless explicitly stated in the claims, the order of processing elements and sequences described in the present disclosure, the use of numerical or alphabetical designations, or the employment of other identifiers, is not intended to limit the sequence of procedures and methods in the present disclosure. Although various examples in the foregoing disclosure discuss embodiments of the invention currently considered useful, it should be understood that such details serve solely illustrative purposes, and the appended claims are not limited to the disclosed embodiments. Conversely, the claims are intended to encompass all modifications and equivalent combinations that conform to the essence and scope of the embodiments in the present disclosure. For example, while the system components described above may be implemented through hardware devices, they could also be realized solely through software solutions, such as installing the described system on existing servers or mobile devices.

Similarly, it should be noted that, to simplify the disclosure of the present disclosure and thereby facilitate understanding of one or more embodiments of the invention, descriptions of the embodiments in the present disclosure may sometimes group multiple features into a single embodiment, figure, or description thereof. However, this disclosure method does not imply that the subject matter of the present disclosure requires more features than those explicitly stated in the claims. In fact, the features of the embodiments may be fewer than all features of a single embodiment disclosed above.

In some embodiments, numerical values describing components or properties are used. It should be understood that such numbers used in describing embodiments may in some examples be modified by terms such as “approximately,” “nearly,” or “substantially.” Unless otherwise specified, “approximately,” “nearly,” or “substantially” indicates that the stated number allows for a variation of +20%. Accordingly, in some embodiments, numerical parameters used in the present disclosure and claims are approximations that may vary depending on the characteristics required for individual embodiments. In some embodiments, numerical parameters should consider the prescribed number of significant digits and employ ordinary rounding techniques. Although numerical ranges and parameters identifying broad scopes in some embodiments of the present disclosure are approximations, in specific embodiments, such numerical values are set as precisely as practicable within feasible extents.

With respect to each patent, patent application, patent application publication, and other materials cited in the present disclosure, such as articles, books, specifications, publications, documents, etc., their entire contents are hereby incorporated into the present disclosure by reference. Excluded are prosecution history documents inconsistent or conflicting with the content of the present disclosure, as well as documents that limit the broadest scope of the claims in the present disclosure (whether currently or subsequently appended to the present disclosure). It should be noted that if descriptions, definitions, and/or terminology usage in the incorporated materials conflict with those in the present disclosure, the descriptions, definitions, and/or terminology usage in the present disclosure shall prevail.

Finally, it should be understood that the embodiments described in the present disclosure are solely intended to illustrate the principles of the embodiments of the present disclosure. Other variations may also fall within the scope of the present disclosure. Therefore, by way of example and not limitation, alternative configurations of the embodiments of the present disclosure may be regarded as consistent with the teachings of the present disclosure. Accordingly, the embodiments of the present disclosure are not limited to those explicitly introduced and described herein.

What is claimed is:

1. A system for estimating the biological age of a subject, comprising:
at least one storage device configured to store a set of instructions; and
at least one processor operatively coupled to the at least one storage device, wherein when executing the set of instructions, the at least one processor is configured to perform operations, the operations comprise:
obtaining quantified abundances of one or more target metabolites from a plurality of metabolites in a biological sample derived from a subject via a quantitative measurement device, wherein the plurality of target metabolites comprises metabolites enumerated in Table A:
TABLE A

| Number | Ion Mode | m/z | Metabolite | Exact Mass | Adduct Ion | Δ(ppm) |
|---|---|---|---|---|---|---|
| MET001 | Negative | 369.1743 | C20H22O3 | 310.15689 | M + Hac − H | 10 |
| MET002 | Negative | 369.1743 | C18H28O3S | 324.17592 | M + FA − H | 0 |
| MET003 | Negative | 369.1712 | C21H24O3 | 324.17254 | M + FA − H | 1 |
| MET004 | Negative | 371.1867 | C22H28O5 | 372.19367 | M − H | 1 |
| MET005 | Negative | 465.2453 | C25H38O8 | 466.25667 | M − H | 9 |
| MET006 | Positive | 431.3104 | C27H42O4 | 430.30831 | M + H | 12 |
| MET007 | Negative | 502.2896 | C25H46NO7P | 503.30119 | M − H | 9 |
| MET008 | Negative | 369.1743 | C22H26O5 | 370.17802 | M − H | 10 |
| MET009 | Positive | 247.1065 | C13H14N2O3 | 246.10044 | M + H | 5 |
| MET010 | Positive | 280.1540 | C15H21NO4 | 279.14706 | M + H | 1 |
| MET011 | Positive | 265.1178 | C13H16N2O4 | 264.11101 | M + H | 2 |
| MET012 | Negative | 151.0251 | C5H4N4O2 | 152.03343 | M − H | 7 |
| MET013 | Positive | 357.2781 | C24H36O2 | 356.27153 | M + H | 2 |
| MET014 | Negative | 369.1743 | C19H30O5S | 370.18140 | M − H | 1 |
| MET015 | Positive | 504.3076 | C23H43O7P | 462.27464 | M + ACN + H | 2; |

based on the quantified abundance of each of the one or more target metabolites, the biological age of the subject is estimated by utilizing a predictive model.

2. The system according to claim 1, wherein the one or more target metabolites comprise at least two, three, or ten metabolites from Table A.

3. The system according to claim 1, wherein the one or more target metabolites comprise all metabolites listed in Table A.

4. The system according to claim 1, wherein the plurality of target metabolites further comprise metabolites listed in Table B:
TABLE B

| | Ion | | | Exact | Adduct | |
|---|---|---|---|---|---|---|
| Number | Mode | m/z | Metabolite | Mass | Ion | Δ(ppm) |
| MET016 | Positive | 287.0989 | C16H14O5 | 286.08412 | M + H | 26 |
| MET017 | Positive | 286.1432 | C17H19NO3 | 285.13649 | M + H | 2 |
| MET018 | Negative | 526.3474 | C24H52NO6P | 481.35323 | M + FA − H | 8 |
| MET019 | Negative | 367.1555 | C21H24N2O2S | 368.15585 | M − H | 19 |
| MET020 | Negative | 371.1901 | C19H32O5S | 372.19705 | M − H | 1 |
| MET021 | Negative | 365.1356 | C17H22N2O7 | 366.14270 | M − H | 1 |
| MET022 | Positive | 347.1219 | C13H25O7P | 324.13379 | M + Na | 3 |
| MET023 | Negative | 415.2165 | C24H32O6 | 416.21989 | M − H | 9 |
| MET024 | Negative | 567.3128 | C30H49O8P | 568.31651 | M − H | 6 |
| MET025 | Negative | 111.0074 | C6H5Cl | 112.00798 | M − H | 60 |
| MET026 | Negative | 371.1901 | C18H30O3S | 326.19157 | M + FA − H | 1 |
| MET027 | Positive | 288.2892 | C17H37NO2 | 287.28243 | M + H | 2 |
| MET028 | Positive | 263.1386 | C14H18N2O3 | 262.13174 | M + H | 2 |
| MET029 | Positive | 286.1433 | C17H18O4 | 286.12051 | M + NH4 − H2O | 0 |
| MET030 | Positive | 248.0696 | C8H13N3O4S | 247.06268 | M + H | 2 |
| MET031 | Negative | 203.0820 | C11H12N2O2 | 204.08988 | M − H | 3 |
| MET032 | Positive | 312.1577 | C19H21NO3 | 311.15214 | M + H | 6 |
| MET033 | Positive | 130.0498 | C5H7NO3 | 129.04259 | M + H | 1 |
| MET034 | Negative | 437.0513 | C19H19ClN2O6S | 438.06524 | M − H | 15 |
| MET035 | Negative | 528.2592 | C26H43NO8S | 529.27094 | M − H | 8 |
| MET036 | Positive | 146.0598 | C9H7NO | 145.05276 | M + H | 2 |
| MET037 | Negative | 191.0190 | C6H8O7 | 192.02700 | M − H | 4 |
| MET038 | Positive | 309.0211 | C6H14O10P2 | 308.00622 | M + H | 25 |
| MET039 | Negative | 194.0452 | C9H9NO4 | 195.05316 | M − H | 3 |
| MET040 | Negative | 399.2214 | C24H32O5 | 400.22497 | M − H | 9 |
| MET041 | Positive | 114.0915 | C6H11NO | 113.08406 | M + H | 1 |
| MET042 | Positive | 450.3207 | C21H45NO3S | 391.31202 | M + Hac − H | 11 |
| MET043 | Negative | 173.0923 | C8H14O4 | 174.08921 | M − H | 60 |
| MET044 | Positive | 205.0969 | C9H9NO2 | 163.06333 | M + ACN + H | 1 |
| MET045 | Positive | 363.2158 | C20H26O4 | 330.18311 | M + CH3OH + H | 2. |

5. The system according to claim 4, wherein the one or more target metabolites comprise at least one metabolite from Table A and at least one metabolite from Table B.

6. The system according to claim 4, wherein the one or more target metabolites comprise at least one metabolite from Table A and at least two metabolites from Table B.

7. The system according to claim 4, wherein the one or more target metabolites comprise two metabolites from Table A and one metabolite from Table B.

8. The system according to claim 1, wherein the plurality of target metabolites further comprise metabolites from Table C:
TABLE C

| | Ion | | | Exact | Adduct | |
|---|---|---|---|---|---|---|
| Number | Mode | m/z | Metabolite | Mass | Ion | Δ(ppm) |
| MET046 | Negative | 367.1580 | C14H26O8 | 322.16277 | M + FA − H | 8 |
| MET047 | Negative | 399.2214 | C21H36O5S | 400.22835 | M − H | 1 |
| MET048 | Negative | 447.3120 | C21H40O6 | 388.28249 | M + Hac − H | 35 |
| MET049 | Positive | 398.3250 | C21H40O4 | 356.29266 | M + ACN + H | 4 |
| MET050 | Negative | 447.3120 | C27H44O5 | 448.31887 | M − H | 1 |
| MET051 | Negative | 447.3120 | C26H42O3 | 402.31340 | M + FA − H | 1 |
| MET052 | Positive | 442.3520 | C25H47NO5 | 441.34542 | M + H | 2 |
| MET053 | Negative | 369.1740 | C15H24N4O4 | 324.17976 | M + FA − H | 11 |
| MET054 | Negative | 367.1587 | C19H28O5S | 368.16575 | M − H | 1 |
| MET055 | Positive | 500.1700 | C26H29NO9 | 499.18423 | M + H | 43 |
| MET056 | Negative | 361.2020 | C21H30O5 | 362.20932 | M − H | 0 |
| MET057 | Negative | 319.2280 | C20H32O3 | 320.23515 | M − H | 0 |
| MET058 | Positive | 398.3260 | C23H43NO4 | 397.31921 | M + H | 1 |
| MET059 | Negative | 511.3020 | C31H44O6 | 512.31379 | M − H | 9 |
| MET060 | Negative | 448.3070 | C26H43NO5 | 449.31412 | M − H | 0 |
| MET061 | Negative | 297.9830 | C5H7NO6P2 | 238.97486 | M + Hac − H | 19 |
| MET062 | Positive | 372.3000 | C24H37NO2 | 371.28243 | M + H | 28 |
| MET063 | Negative | 173.1190 | C9H18O3 | 174.12559 | M − H | 4 |
| MET064 | Positive | 341.2320 | C19H32O5 | 340.22497 | M + H | 1 |
| MET065 | Negative | 590.3460 | C33H45N5O5 | 591.34207 | M − H | 19 |
| MET066 | Positive | 302.1960 | C16H23N5O | 301.19026 | M + H | 5 |
| MET067 | Negative | 314.1030 | C17H17NO5 | 315.11067 | M − H | 1 |
| MET068 | Positive | 355.2270 | C23H30O3 | 354.21950 | M + H | 1 |
| MET069 | Positive | 181.0720 | C7H10N4O3 | 198.07529 | M + H − H2O | 3 |
| MET070 | Positive | 180.0647 | C7H8N4O2 | 180.06473 | M+ | 0 |
| MET071 | Negative | 504.3100 | C32H43NO4 | 505.31921 | M − H | 4 |
| MET072 | Negative | 397.2057 | C23H30N2O4 | 398.22056 | M − H | 19 |
| MET073 | Positive | 355.2830 | C21H38O4 | 354.27701 | M + H | 4 |
| MET074 | Positive | 195.0870 | C8H10N4O2 | 194.08038 | M + H | 3 |
| MET075 | Negative | 427.1630 | C24H23F3N2O2 | 428.17116 | M − H | 2 |
| MET076 | Positive | 432.3110 | C25H41NO2 | 387.31373 | M + FA − H | 2 |
| MET077 | Negative | 415.2165 | C16H34O9 | 370.22028 | M + FA − H | 5; |

wherein the one or more target metabolites comprise at least one metabolite from Table A and at least one metabolite from Table C.

9. The system according to claim 1, wherein the quantified abundance of each of the one or more target metabolites is determined by the quantitative measurement device using a relative quantification method or an absolute quantification method.

10. The system according to claim 1, wherein the prediction model processes the quantified abundance of each of the one or more target metabolites to determine a sample score.

11. The system according to claim 10, wherein the sample score indicates the biological age of the subject.

12. The system according to claim 1, wherein the prediction model is a trained machine learning model.

13. The system according to claim 12, wherein the trained machine learning model is obtained by training a preliminary model using a plurality of training datasets, wherein
each of the plurality of training datasets comprises the quantified abundance of the one or more target metabolites from a reference sample of a reference subject, and a label indicating the biological age of the reference subject.

14. The system according to claim 1, wherein the quantitative measurement device is a liquid chromatography-mass spectrometry (LC-MS) system.

15. A method for estimating the physiological age of a subject, comprising:
(a) obtaining quantified abundances of one or more target metabolites from a sample of the subject via a quantitative measurement device, wherein the target metabolites comprise metabolites listed in Table A;
(b) estimating the physiological age of the subject using a predictive model based on the quantified abundances of each of the one or more target metabolites.

16. The method according to claim 15, wherein the target metabolites further comprise metabolites listed in Table B, wherein the one or more target metabolites include at least one metabolite from Table A and at least one metabolite from Table B.

17. The method according to claim 15, wherein the target metabolites further comprise metabolites listed in Table C, wherein the one or more target metabolites include at least one metabolite from Table A and at least one metabolite from Table C.

18. A kit for estimating the physiological age of a subject, comprising one or more target metabolites from a group of multiple metabolites, wherein the target metabolites comprise metabolites listed in Table A.

19. The kit according to claim 18, wherein the target metabolites further comprise metabolites listed in Table B, wherein the one or more target metabolites include at least one metabolite from Table A and at least one metabolite from Table B.

20. The kit according to claim 18, wherein the target metabolites further comprise metabolites listed in Table C, wherein the one or more target metabolites include at least one metabolite from Table A and at least one metabolite from Table C.
